(19) United States
(12) Patent Application Publication
(10) Pub. No.: US 2026/0207738 A1
(43) Pub. Date: Jul. 23, 2026

(54) COMPOSITIONS COMPRISING ANTI-CD38 ANTIBODIES AND CARFILZOMIB

(71) Applicants: Sanofi-Aventis U.S, LLC, Morristown, NJ (US); The Regents of the University of California, Oakland, CA (US)

(72) Inventors: Blake TOMKINSON, Bridgewater, NJ (US); Blake T. AFTAB, San Francisco, CA (US); Byron C. HANN, San Francisco, CA (US); Thomas G. MARTIN III, San Francisco, CA (US)

(21) Appl. No.: 19/417,983

(22) Filed: Dec. 12, 2025

Related U.S. Application Data
(63) Continuation of application No. 17/324,478, filed on May 19, 2021, now Pat. No. 12,533,411.
Application No. 17/324,478 is a continuation of application No. 16/375,693, filed on Apr. 4, 2019, now abandoned.
Application No. 16/375,693 is a continuation of application No. 15/389,279, filed on Dec. 22, 2016, now abandoned.
Application No. 15/389,279 is a continuation of application No. 14/775,641, filed on Mar. 13, 2014, now abandoned.
(60) Provisional application No. 61/778,540, filed on Mar. 13, 2013.
(60) Provisional application No. 61/808,381, filed on Apr. 4, 2013.

(51) Int. Cl. A61K 39/395; A61K 9/00; A61K 31/573; A61K 38/07; A61K 38/08; A61K 39/00; C07K 16/28; C07K 16/30

(52) U.S. Cl. CPC A61K 39/39533; A61K 9/0019; A61K 9/0053; A61K 31/573; A61K 38/07; A61K 38/08; A61K 39/39558; C07K 16/2896; C07K 16/3061; A61K 2039/505; A61K 2039/54; A61K 2039/545; C07K 2317/24; C07K 2317/56; C07K 2317/73; C07K 2317/732; C07K 2317/734

(57) ABSTRACT

Disclosed herein are compositions and kits which comprise anti-CD38 antibodies and carfilzomib compounds. Also disclosed are methods for treating cancers, such as multiple myeloma, in subjects with the compositions and kits.

### CROSS-REFERENCE TO RELATED APPLICATIONS

This application is a continuation of U.S. patent application Ser. No. 17/324,478 (now U.S. Pat. No. 12,533,411), filed May 19, 2021, which is a continuation of U.S. patent application Ser. No. 16/375,693 (now abandoned), filed Apr. 4, 2019, which is a continuation of U.S. patent application Ser. No. 15/389,279 (now abandoned), filed Dec. 22, 2016, which is a continuation of U.S. patent application Ser. No. 14/775,641 (now abandoned), which adopts the international filing date of Mar. 13, 2014, and is a National Phase application under 35 U.S.C. § 371 of International Application No. PCT/US2014/025441, filed Mar. 13, 2014, which claims the priority benefit of U.S. Provisional Application No. 61/778,540, filed Mar. 13, 2013, and U.S. Provisional Application No. 61/808,381, filed Apr. 4, 2013, the disclosures of which are herein incorporated by reference in their entirety.

### SUBMISSION OF SEQUENCE LISTING

The contents of the electronic Sequence Listing (file name: 183952000704SEQLIST.xml, size: 89,819 bytes; and Date of Creation: Nov. 24, 2025) is herein incorporated by reference in its entirety.

### BACKGROUND OF THE INVENTION

### 1. Field of the Invention

The field of the present invention relates to anti-CD38 antibodies, carfilzomib, and cancer treatments.

### 2. Description of the Related Art

Multiple myeloma (MM) is a B cell malignancy. In MM, abnormal plasma cells accumulate in the bone marrow where they interfere with the production of normal cells. Current therapy of MM includes administration of proteasome inhibitors such as bortezomib and carfilzomib, immunomodulatory drugs such as lenalidomide and thalidomide, and chemotherapy such as melphalan and prednisone. While these agents have improved survival in multiple myeloma, invariably resistance becomes problematic and patients succumb from their illness. Multiple myeloma thus remains ultimately fatal, with a median survival of approximately 3 to 5 years only.

CD38 is expressed on malignant plasma cells. CD38 is a 45 kD type II transmembrane glycolprotein with a long C-terminal extracellular domain and a short N-terminal cytoplasmic domain. The CD38 protein is a bifunctional ectoenzyme that can catalyze the conversion of NAD⁺ into cyclic ADP-ribose (cADPR) and also hydrolyze cADPR into ADP-ribose. CD38 is up-regulated and has been implicated in many hematopoietic malignancies.

Thus, some proposed MM treatments include the administration of anti-CD38 antibodies. See, for example, WO 2012/041800; de Weers et al. (2011) J Immunol 186:1840-1848; and Van der Veer et al. (2011) Haematologica 96(2):284-290. Unfortunately, like various drugs and chemotherapies, not all antibodies are the same and not all antibodies against the same antigen exhibit the same activities.

There is thus a need for new and efficacious treatments for extending survival and improving outcome of treatments of multiple myeloma, and more generally of blood cancers.

### DESCRIPTION OF THE DRAWINGS

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

This invention is further understood by reference to the drawings wherein:

FIG. 1A shows the growth rate of tumors in xenograft models implanted with NCI-H929 cells (H929 models).

FIG. 1B shows the growth rate of tumors in xenograft models implanted with RPMI 8226 cells (RPMI models).

FIG. 2A shows the tumor volume of tumors in RPMI models after treatment with the indicated dose of hu38SB19 at the indicated times (arrows).

FIG. 2B shows the body weight of the RPMI models after treatment with the indicated dose of hu38SB19 at the indicated times (arrows).

FIG. 3A shows the tumor volume of tumors in H929 models after treatment with the indicated dose of hu38SB19 at the indicated times (arrows).

FIG. 3B shows the body weight of the H929 models after treatment with the indicated dose of hu38SB19 at the indicated times (arrows).

FIG. 4A shows the tumor volume of tumors in H929 models after treatment with the indicated dose of hu38SB19 at the indicated times (arrows).

FIG. 4B shows the body weight of the H929 models after treatment with the indicated dose of hu38SB19 at the indicated times (arrows).

FIG. 5A shows the tumor volume of tumors in H929 models after treatment with the indicated dose of hu38SB19 at the indicated times (arrows).

FIG. 5B shows the body weight of the H929 models after treatment with the indicated dose of hu38SB19 at the indicated times (arrows).

FIG. 6A shows the tumor volume of tumors in H929 models after treatment with the indicated dose of carfilzomib at the indicated times (arrows).

FIG. 6B shows the body weight of the H929 models after treatment with the indicated dose of carfilzomib at the indicated times (arrows).

FIG. 7A shows the tumor volume of tumors in RPMI models after treatment with the indicated dose of carfilzomib at the indicated times (arrows).

FIG. 7B shows the body weight of the RPMI models after treatment with the indicated dose of carfilzomib at the indicated times (arrows).

FIG. 8A shows the tumor volume of tumors in H929 models after treatment with the indicated dose of hu38SB19 at the indicated times (top arrows) and the indicated dose of carfilzomib at the indicated times (bottom arrows).

FIG. 8B shows the body weight of the H929 models after treatment with the indicated dose of hu38SB19 at the indicated times (top arrows) and the indicated dose of carfilzomib at the indicated times (bottom arrows).

FIG. 9A is a graph showing the mean wet tumor weights of the H929 models after the indicated treatment with carfilzomib and/or hu38SB19 (mAb).

FIG. 9B is a graph showing the median wet tumor weights of the H929 models atter the indicated treatment with carfilzomib and/or hu38SB19 (mAb).

FIG. 10A shows the tumor volume of tumors in RPMI-8226 models after treatment with the indicated dose of hu38SB19 at the indicated times (top arrows) and the indicated dose of carfilzomib at the indicated times (bottom arrows).

FIG. 10B shows the body weight of the RPMI-8226 models after treatment with the indicated dose of hu38SB19 at the indicated times (top arrows) and the indicated dose of carfilzomib at the indicated times (bottom arrows).

FIG. 11 is a graph showing the cell surface density of CD38 in multiple myeloma cell lines.

FIG. 12 is a graph showing that hu38SB19, as the sole active ingredient, results in dose-dependent anti-tumor effects and eradication of NCI-H929 hind-flank xenograft tumor growth. Four cumulative doses, given twice weekly at 5 mg/kg were sufficient to eliminate palpable tumors in all mice within the cohort.

FIG. 13 is a graph showing that low-dose combinations of carfilzomib and hu38SB19 results in near complete tumor growth inhibition of NCI-H929 xenografts.

### SUMMARY OF THE INVENTION

In some embodiments, the present invention relates to a method of treating a cancer in a subject which comprises administering one or more anti-CD38 antibodies and one or more carfilzomib compounds to the subject. In some embodiments, the cancer is a hematological malignancy. In some embodiments, the cancer is multiple myeloma. In some embodiments, the cancer is a relapsed multiple myeloma or a refractory multiple myeloma. In some embodiments, the one or more carfilzomib compounds is carfilzomib. In some embodiments, the one or more anti-CD38 antibodies are administered in an effective amount, preferably a synergistic amount. In some embodiments, the one or more anti-CD38 antibodies and/or the one or more carfilzomib compounds are administered in a therapeutically effective amount. In some embodiments, at least one of the one or more anti-CD38 antibodies is capable of killing a CD38+ cell by apoptosis, antibody-dependent cell-mediated cytotoxicity (ADCC), and complement-dependent cytotoxicity (CDC). In some embodiments, the antibody is hu38SB19. In some embodiments, at least one of the one or more anti-CD38 antibodies comprises one or more complementarity-determining region having an amino acid sequence selected from the group consisting of SEQ ID NOs: 13, 14, 81, 15, 16, 17, 18, 1, 2, 3, 4, 5, 6, 7, 8, 9, 10, 11, 12, 19, 20, 21, 22, 23, 24, 25, 26, 27, 28, 29, 30, 31, 32, 33, 34, 35 and 36. In some embodiments, at least one of the one or more anti-CD38 antibodies is selected from the group consisting of: a) an antibody comprising a heavy chain comprising three sequential CDRs having amino acid sequences consisting of SEQ ID NOs: 13, 15 and either SEQ ID NO: 14 or SEQ ID NO: 81, and a light chain comprising three sequential CDRs having amino acid sequences consisting of SEQ ID NOs: 16, 17 and 18; b) an antibody comprising a heavy chain comprising three sequential CDRs having amino acid sequences consisting of SEQ ID NOs: 25, 26 and 27, and a light chain comprising three sequential CDRs having amino acid sequences consisting of SEQ ID NOs: 28, 29 and 30; c) an antibody comprising a heavy chain comprising three sequential CDRs having amino acid sequences consisting of SEQ ID NOs: 1, 2 and 3, and a light chain comprising three sequential CDRs having amino acid sequences consisting of SEQ ID NOs: 4, 5 and 6; d) an antibody comprising a heavy chain comprising three sequential CDRs having amino acid sequences consisting of SEQ ID NOs: 7, 8 and 9, and a light chain comprising three sequential CDRs having amino acid sequences consisting of SEQ ID NOs: 10, 11 and 12; e) an antibody comprising a heavy chain comprising three sequential CDRs having amino acid sequences consisting of SEQ ID NOs: 19, 20 and 21, and a light chain comprising three sequential CDRs having amino acid sequences consisting of SEQ ID NOs: 22, 23 and 24; and f) an antibody comprising a heavy chain comprising three sequential CDRs having amino acid sequences consisting of SEQ ID NOs: 31, 32 and 33, and a light chain comprising three sequential CDRs having amino acid sequences consisting of SEQ ID NOs: 34, 35 and 36. In some embodiments, the antibody comprises a heavy chain having a VH variable region represented by SEQ ID NO: 66, and a light chain having a VL variable region represented by either SEQ ID NO: 62 or SEQ ID NO: 64. In some embodiments, the antibody comprises a heavy chain having a VH variable region represented by SEQ ID NO: 72, and a light chain having a VL variable region represented by either SEQ ID NO: 68 or SEQ ID NO: 70. In some embodiments, the one or more anti-CD38 antibodies are administered intravenously. In some embodiments, the one or more carfilzomib compounds are administered orally. In some embodiments, the one or more anti-CD38 antibodies and the one or more carfilzomib compounds are administered sequentially. In some embodiments, the method further comprises administering a dexamethasone compound, preferably dexamethasone, to the subject. In some embodiments, the dexamethasone compound is administered orally. In some embodiments, the dexamethasone compound is administered at a low dose. In some embodiments, the one or more anti-CD38 antibodies, the one or more carfilzomib compounds, and the dexamethasone compound are administered sequentially. In some embodiments, the one or more anti-CD38 antibodies and the one or more carfilzomib compounds are administered sequentially.

In some embodiments, the present invention relates to a composition comprising a) at least one anti-CD38 antibody, preferably the antibody is capable of killing a CD38+ cell by apoptosis, antibody-dependent cell-mediated cytotoxicity (ADCC), and complement-dependent cytotoxicity (CDC); and b) at least one carfilzomib compound, preferably carfilzomib; and, optionally c) a dexamethasone compound, preferably dexamethasone. In some embodiments, the present invention relates to a composition comprising a) at least one anti-CD38 antibody; and b) at least one carfilzomib compound; and, optionally i) a dexamethasone compound. In some embodiments, the antibody is capable of killing a CD38+ cell by apoptosis, antibody-dependent cell-mediated cytotoxicity (ADCC), and complement-dependent cytotoxicity (CDC). In some embodiments, the antibody is hu38SB19. In some embodiments, the carfilzomib compound is carfilzomib. In some embodiments, the dexamethasone compound is dexamethasone.

In some embodiments, the present invention is directed to a kit comprising a) a first composition comprising at least one anti-CD38 antibody, preferably the antibody is capable of killing a CD38+ cell by apoptosis, antibody-dependent cell-mediated cytotoxicity (ADCC), and complement-dependent cytotoxicity (CDC); and b) a second composition comprising at least one carfilzomib compound, preferably carfilzomib. In some embodiments, the compositions in the kit are packaged for sequential administration to a subject. In some embodiments, the antibody is hu38SB19. In some embodiments, the kit further includes a dexamethasone compound, preferably dexamethasone. In some embodiments, the carfilzomib compound and the dexamethasone compound are packaged for sequential administration to a subject.

In some embodiments, the present invention is directed to a kit comprising at least one anti-CD38 antibody capable of killing a CD38+ cell by apoptosis, antibody-dependent cell-mediated cytotoxicity (ADCC), and complement-dependent cytotoxicity (CDC), packaged together with a label having one or more messages that the at least one anti-CD38 antibody shall be administered in combination with carfilzomib, and optionally with dexamethasone. In some embodiments, the antibody is hu38SB19. In some embodiments, the kit further includes a dexamethasone compound, preferably dexamethasone. In some embodiments, the carfilzomib compound and the dexamethasone compound are packaged for sequential administration to a subject.

In some embodiments, the present invention is directed to a combination of: (i) at least one anti-CD38 antibody, preferably the antibody is capable of killing a CD38+ cell by apoptosis, antibody-dependent cell-mediated cytotoxicity (ADCC), and complement-dependent cytotoxicity (CDC); and (ii) at least one carfilzomib compound, preferably carfilzomib; and, optionally (iii) a dexamethasone compound, preferably dexamethasone. In some embodiments, the present invention relates to a combination comprising a) at least one anti-CD38 antibody; and b) at least one carfilzomib compound; and, optionally i) a dexamethasone compound. In some embodiments, the antibody is capable of killing a CD38+ cell by apoptosis, antibody-dependent cell-mediated cytotoxicity (ADCC), and complement-dependent cytotoxicity (CDC). In some embodiments, the antibody is hu38SB19. In some embodiments, the carfilzomib compound is carfilzomib. In some embodiments, the dexamethasone compound is dexamethasone. In some embodiments, the combination is for sequential use in the treatment of a hematological malignancy, preferably multiple myeloma.

In some embodiments, the present invention is directed to use of (i) at least one anti-CD38 antibody, preferably the antibody is capable of killing a CD38+ cell by apoptosis, antibody-dependent cell-mediated cytotoxicity (ADCC), and complement-dependent cytotoxicity (CDC); and (ii) at least one carfilzomib compound, preferably carfilzomib; and, optionally (iii) a dexamethasone compound, preferably dexamethasone, for the treatment of a hematological malignancy, preferably multiple myeloma. In some embodiments, the present invention relates to use of a) at least one anti-CD38 antibody; and b) at least one carfilzomib compound; and, optionally i) a dexamethasone compound, for the treatment of a hematological malignancy, preferably multiple myeloma. In some embodiments, the antibody is capable of killing a CD38+ cell by apoptosis, antibody-dependent cell-mediated cytotoxicity (ADCC), and complement-dependent cytotoxicity (CDC). In some embodiments, the antibody is hu38SB19. In some embodiments, the carfilzomib compound is carfilzomib. In some embodiments, the dexamethasone compound is dexamethasone.

In some of the various embodiments of the present invention, the subject to be treated is mammalian. In some of the various embodiments of the present invention, the subject to be treated is a test animal such as a mouse. In some of the various embodiments of the present invention, the subject to be treated is human.

### DETAILED DESCRIPTION OF THE INVENTION

The present invention relates to methods of treating a cancer in a subject which comprises administering one or more anti-CD38 antibodies and one or more carfilzomib compounds to the subject. As used herein, “treat” or “treating” means to alleviate symptoms, eliminate the causation of the symptoms either on a temporary or permanent basis, or to prevent or slow the appearance of symptoms of the named disorder or condition. As disclosed herein, the efficacy of a carfilzomib compound is considerably improved when administered in conjunction with one or more anti-CD38 antibodies according to the present invention. In fact, the administration of one or more anti-CD38 antibodies which exhibit (a) the capability of killing a CD38⁺ cell by apoptosis, (b) antibody-dependent cell-mediated cytotoxicity (.ADCC), and (c) complement-dependent cytotoxicity (CDC) is believed to considerably improve the efficacy of carfilzomib compounds in the treatment of hematological malignancies, including MM, to a degree that is unexpectedly more than other anti-CD38 antibodies which do not exhibit all three (a)-(c) activities. Therefore, in some embodiments, the one or more anti-CD38 antibodies are capable of (a) killing a CD38⁺ cell by apoptosis, (b) antibody-dependent cell-mediated cytotoxicity (ADCC), and (c) complement-dependent cytotoxicity (CDC). In some embodiments, the one or more anti-CD38 antibodies and/or the one or more carfilzomib compounds are administered in a therapeutically effective amount. As used herein, a “therapeutically effective amount” of a substance refers to an amount of that substance that results in the alleviation of one or more symptoms, elimination of the causation of the symptoms either on a temporary or permanent basis, and/or the prevention or reduction in the appearance of symptoms of the named disorder or condition in the majority of subjects afflicted with and similarly treated for the named disease or disorder.

In some embodiments, the cancer is one in which CD38 is expressed by the malignant cells. In some embodiments, the cancer is a hematological malignancy of the blood, bone marrow, and/or lymph nodes. In some embodiments, the cancer is a blood cancer. Blood cancers include myeloma, lymphoma and leukemia. The blood cancer might, for instance, be selected from the group consisting of multiple myeloma, non-Hodgkin's lymphoma, Hodgkin's lymphoma, hairy cell leukemia, chronic lymphocytic leukemia, chronic myeloid leukemia, acute myeloid leukemia, and acute lymphocytic leukemia. In some embodiments, the cancer is multiple myeloma (MM). In some embodiments, the cancer is a relapse MM or refractory MM. As used herein, relapsed MM refers to clinically active MM after a period of remission and refractory MM refers to progressive or stable disease while being treated or progressive disease within 3 months of the last does of the prior treatment. See Dimopoulos et al. (2010) Eur J Haematology 88:1-15.

In some embodiments, the subject is mammalian, preferably human. In some embodiments, the subject is an adult human, e.g., at least 18 years. In some embodiments, the subject is in need of treatment for the cancer. In some embodiments, the subject has been diagnosed as having the cancer. In some embodiments, the cancer is in partial or complete remission, however, the one or more carfilzomib compounds and the one or more anti-CD38 antibodies are administered to the subject so as to reduce the likelihood of relapse. In some embodiments, the subject has a Karnofsky performance status equal or superior to 60%. The Karnofsky status runs from 100 to 0, where 100 is “perfect” health and 0 is death (Karnofsky and Burchenal, 1949, “The Clinical Evaluation of Chemotherapeutic Agents in Cancer.” In: MacLeod CM (Ed), Evaluation of Chemotherapeutic Agents. Columbia Univ Press). In some embodiments, the subject has undergone at least one or two prior therapies for multiple myeloma, induction therapy being considered one prior therapy. In some embodiments, the subject exhibits evidence that either the cancer progressed while the subject underwent a prior therapy, or that the subject was refractory to the prior therapy.

In some embodiments, the anti-CD38 antibodies specifically bind CD38. In some embodiments, the anti-CD38 antibodies are raised against CD38 or an epitope thereof. In some embodiments, the anti-CD38 antibodies are monoclonal antibodies. In some embodiments, one or more of the anti-CD38 antibodies according to the present invention are monoclonal antibodies as described in WO 2008/047242, which is herein incorporated by reference in its entirety. In some embodiments, one or more of the anti-CD38 antibodies are monoclonal antibodies 38SB13, 38SB18, 38SB19, 38SB30, 38SB31, and 38SB39 as described in WO 2008/047242, which is herein incorporated by reference in its entirety. In some embodiments, the one or more anti-CD38 antibodies are capable of killing CD38⁺ cells by three different cytotoxic mechanisms, induction of apoptosis, antibody-dependent cell-mediated cytotoxicity (ADCC), and complement-dependent cytotoxicity (CDC).

The term “antibody” is used herein in the broadest sense and includes monoclonal antibodies (including full length monoclonal antibodies) of any isotype such as IgG, IgM, IgA, IgD and IgE, polyclonal antibodies, multispecific antibodies, chimeric antibodies, and antibody fragments. As used herein, the prefix “anti-” when in conjunction with an antigen, indicates that the given antibody is reactive with the given antigen. An antibody reactive with a specific antigen can be generated by synthetic and/or recombinant methods such as selection of libraries of recombinant antibodies in phage or similar vectors, or by immunizing an animal with the antigen or an antigen-encoding nucleic acid.

A typical IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are usually referred to as CDR1, CDR2, and CDR3, numbered sequentially from the N-terminus. The more highly conserved portions of the variable regions outside of the CDRs are called the “framework regions”. As used herein, “V_{H}” or “VH” refers to the variable region of an immunoglobulin heavy chain of an antibody, including the heavy chain of an Fv, scFv, dsFv, Fab, Fab′ or F(ab′)2 fragment. Reference to “V_{L}” or “VL” refers to the variable region of the immunoglobulin light chain of an antibody, including the light chain of an Fv, scFv, dsFv, Fab, Fab′ or F(ab′)2 fragment.

The antibodies according to the present invention may be, e.g., murine, chimeric, and/or humanized antibodies. As used herein, a “chimeric antibody” is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Methods for producing chimeric antibodies are known in the art. See e.g., Morrison, 1985, Science, 229:1202; Oi et al., 1986, BioTechniques, 4:214; Gillies et al., 1989, J. Immunol. Methods, 125:191-202; U.S. Pat. Nos. 5,807,715; 4,816,567; and 4,816,397, which are incorporated herein by reference in their entireties. The term “humanized antibody”, as used herein, refers to a chimeric antibody which contain minimal sequence derived from non-human immunoglobulin. The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modelling, statistical analysis and mutagenesis to alter the non-CDR surfaces of antibody variable regions to resemble the surfaces of known antibodies of the target host. The CDR grafting technology involves substituting the complementarity determining regions of, for example, a mouse antibody, into a human framework domain, e.g., see WO 92/22653. Humanized chimeric antibodies preferably have constant regions and variable regions other than the complementarity determining regions (CDRs) derived substantially or exclusively from the corresponding human antibody regions and CDRs derived substantially or exclusively from a mammal other than a human.

Strategies and methods for the resurfacing of antibodies, and other methods for reducing immunogenicity of antibodies within a different host, are disclosed in U.S. Pat. No. 5,639,641, which is hereby incorporated in its entirety by reference. Antibodies can be humanized using a variety of other techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., 1991, Molecular Immunology 28(4/5): 489-498; Studnicka G. M. et al., 1994, Protein Engineering, 7(6): 805-814; Roguska M. A. et al., 1994, PNAS, 91:969-973), chain shuffling (U.S. Pat. No. 5,565,332), and identification of flexible residues (PCT/US2008/074381). Human antibodies can be made by a variety of methods known in the art including phage display methods. See also U.S. Pat. Nos. 4,444,887, 4,716,111, 5,545,806, and 5,814,318; and international patent application publication numbers WO 98/46645, WO 98/50433, WO 98/24893, WO 98/16654, WO 96/34096, WO 96/33735, and WO 91/10741 (said references incorporated by reference in their entireties).

In some embodiments, one or more of the anti-CD38 antibodies according to the invention are capable of killing a CD38⁺ cell by apoptosis, antibody-dependent cell-mediated cytotoxicity (ADCC), and complement-dependent cytotoxicity (CDC). In some embodiments, one or more of the anti-CD38 antibodies according to the invention are capable of killing said CD38⁺ cells by apoptosis even in the absence of stroma cells or stroma-derived cytokines. These activities can be assessed as described in WO 2008/047242, which is hereby incorporated by reference in its entirety.

In some embodiments according to the invention, one or more anti-CD38 antibodies are selected from the group consisting of 38SB13, 38SB18, 38SB19, 38SB30, 38SB31, 38SB39, and antibodies cross-competing with 38SB13, 38SB18, 38SB19, 38SB30, 38SB31 or 38SB39. The hybridoma cell lines producing the 38SB13, 38SB18, 38SB19, 38SB30, 38SB31, and 38SB39 murine anti-CD38 antibodies have been deposited at the American Type Culture Collection (10801 University Bld, Manassas, VA, 20110-2209, USA), on 21 Jun. 21 2006, under the deposit numbers PTA-7667, PTA-7669, PTA-7670, PTA-7666, PTA-7668, and PTA-7671, respectively (as described in WO 2008/047242, which is herein incorporated by reference in its entirety).

As disclosed herein, references to SEQ ID NOs refers to the sequences set forth in the Sequence Listing submitted herewith and also as recited in WO 2008/047242, which is herein incorporated by reference in its entirety. In some embodiments, the anti-CD38 antibodies according to the present invention may, for instance, comprise a heavy chain comprising three sequential CDRs having amino acid sequences represented by SEQ ID NOs: 1, 2, and 3, and a light chain comprising three sequential CDRs having amino acid sequences represented by SEQ ID NOs: 4, 5, and 6. An example of such an antibody is the 38SB13 antibody, which comprises a heavy chain having a V_{H} variable region represented by SEQ ID NO: 50, and a light chain having a V_{L} variable region represented by SEQ ID NO: 38.

In some embodiments, the anti-CD38 antibodies according to the present invention may, for instance, comprise a heavy chain comprising three sequential CDRs having amino acid sequences represented by SEQ ID NOs: 7, 8, and 9, and a light chain comprising three sequential CDRs having amino acid sequences represented by SEQ ID NOs: 10, 11, and 12. An example of such an antibody is the 38SB18 antibody, which comprises a heavy chain having a V_{H} variable region represented by SEQ ID NO: 52 and a light chain having a V_{L}, variable region represented by SEQ ID NO: 40.

In some embodiments, the anti-CD38 antibodies according to the present invention may, for instance, comprise a heavy chain comprising three sequential CDRs having amino acid sequences represented by SEQ ID NO: 13, SEQ ID NO: 15 and either SEQ ID NO: 14 or SEQ ID NO: 81, and a light chain comprising three sequential CDRs having amino acid sequences represented by SEQ ID NOs: 16, 17, and 18. An example of such an antibody is the 38SB19 antibody, which comprises a heavy chain having a V_{H} variable region represented by SEQ ID NO: 54 and a light chain having a V_{L}, variable region represented by SEQ ID NO: 42. Specific examples of humanized versions of 38SB19 (hu38SB19) include antibodies comprising a heavy chain having a V_{H} variable region represented by SEQ ID NO: 66, and a light chain having a V_{L}. variable region represented by either SEQ ID NO: 62 or SEQ ID NO: 64. hu38SB19 is a humanized anti-CD38 antibody currently undergoing clinical evaluation in CD38-positive hematologic malignancies, including multiple myeloma. Previous and current studies demonstrate that the anti-myeloma activity associated with this agent involve mechanisms of ADCC, and CDC, as well as novel, direct apoptotic and anti-ADP-ribosyl cyclase activity. See Marie-Cécile Wetzel, Céline Nicolazzi, François Vallée, et al. hu38SB19: characterization of a potent phase I humanized anti-CD38 antibody for the treatment of multiple myeloma and other hematologic malignancies. AACR Annual Meeting 2013, Abstract #4735.

In some embodiments, the anti-CD38 antibodies according to the present invention may, for instance, comprise a heavy chain comprising three sequential CDRs having amino acid sequences represented by SEQ ID NOs: 19, 20, and 21, and a light chain comprising three sequential CDRs having amino acid sequences represented by SEQ ID NOs: 22, 23, and 24. An example of such an antibody is the 38SB30 antibody, which comprises a heavy chain having a V_{H} variable region represented by SEQ ID NO: 56 and a light chain having a V_{L}. variable region represented by SEQ ID NO: 44.

In some embodiments, the anti-CD38 antibodies according to the present invention may, for instance, comprise a heavy chain comprising three sequential CDRs having amino acid sequences represented by SEQ ID NOs: 25, 26, and 27, and a light chain comprising three sequential CDRs having amino acid sequences represented by SEQ ID NOs: 28, 29, and 30. An example of such an antibody is the 38SB31 antibody, which comprises a heavy chain having a V_{H} variable region represented by SEQ ID NO: 58 and a light chain having a V_{L}. variable region represented by SEQ ID NO: 46. Specific examples of humanized versions of 38SB31 (hu38SB31) include antibodies comprising a heavy chain having a V_{H} variable region represented by SEQ ID NO: 72, and a light chain having a V_{L}, variable region represented by either SEQ ID NO: 68 or SEQ ID NO: 70.

In some embodiments, the anti-CD38 antibodies according to the present invention may, for instance, comprise a heavy chain comprising three sequential CDRs having amino acid sequences represented by SEQ ID NOs: 31, 32 and 33, and a light chain comprising three sequential CDRs having amino acid sequences represented by SEQ ID NOs: 34, 35, and 36. An example of such an antibody is the 38SB39 antibody, which comprises a heavy chain having a V_{H} variable region represented by SEQ ID NO: 60 and a light chain having a V_{L}. variable region represented by SEQ ID NO: 48.

In some embodiments, the anti-CD38 antibodies according to the invention are humanized antibodies consisting of two identical heavy chains and of two identical light chains, wherein each chain consists of one constant region and of one variable region.

As used herein, a “carfilzomib compound” refers to carfilzomib (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide and carfilzomib derivatives. As used herein, “carfilzomib derivatives” refers to compounds which have 2-acetamido-N-(1-((1-(1-methylcyclopropyl)-1-oxopropan-2-yl)amino)-1-oxopropan-2-yl) propanamide, i.e.,

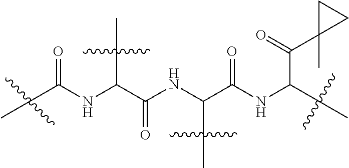 [SMILES: *C(=O)NC(*)C(=O)NC(*)C(=O)NC(*)C(=O)C1(C)CC1]

which may or may not be substituted, as part of its structural formula. In some embodiments, carfilzomib derivatives include compounds which have the following structure, which may or may not be substituted, as part of its structural backbone:

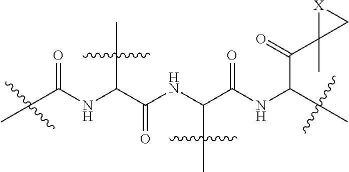

wherein X is selected from O, NH, and N—C₁₋₆alkyl, preferably O. Examples of “carfilzomib derivatives” according to the present invention include those as set forth in U.S. Pat. Nos. 7,232,818; 7,417,042; 7,491,704; 7,737,112; 8,129,346; 8,207,125; 8,207,126; 8,207,127; and 8,207,297.

In some embodiments, the one or more anti-CD38 antibodies are administered in an effective amount. As used herein, an effective amount of the one or more anti-CD38 antibodies is an amount which results in an additive or a synergistic effect with the one or more carfilzomib compounds. As used herein, a “synergistic amount” is one that results in a synergistic effect. As used herein, a “synergistic effect” refers to the effect of the combination of the one or more anti-CD38 antibodies and the one or more carfilzomib compounds which is more than their expected additive effect. In some embodiments, the one or more anti-CD38 antibodies are administered before, during, and/or after the administration of the one or more carfilzomib compounds. In some embodiments, the one or more anti-CD38 antibodies and the one or more carfilzomib compounds are co-administered in the form of a single composition, e.g., as a mixture.

Thus, in some embodiments, the present invention is directed to compositions comprising a mixture of at least one anti-CD38 antibody and at least one carfilzomib compound. In some embodiments, the mixture comprises the at least one anti-CD38 antibody in an amount that results in an additive or a synergistic effect with the at least one carfilzomib compound in a subject when both are administered. In some embodiments, the at least one anti-CD38 antibody in the mixture is one which is capable of killing a CD38⁺ cell by apoptosis, antibody-dependent cell-mediated cytotoxicity (ADCC), and complement-dependent cytotoxicity (CDC); and at least one carfilzomib compound.

For the purposes of the present invention, the methods and compositions of the present invention are not exclusively limited to those which are obtained by physical association of the anti-CD38 antibodies and the carfilzomib compound, but also to those which permit a separate administration, which can be simultaneous or spaced out over a period of time. Thus, in some embodiments, the present invention is directed to a first composition comprising the one or more anti-CD38 antibodies, and a second composition comprising one or more carfilzomib compounds. In some embodiments, the at least one anti-CD38 antibody is one which is capable of killing a CD38⁺ cell by apoptosis, antibody-dependent cell-mediated cytotoxicity (ADCC), and complement-dependent cytotoxicity (CDC); and at least one carfilzomib compound. In some embodiments, the amount of the one or more anti-CD38 antibodies provided in the first composition is one that results in an additive or a synergistic effect with the at least one carfilzomib compound in the second composition in a subject when both are administered.

In some embodiments, the first and second compositions may be packaged in a kit. Thus, in some embodiments, the present invention is directed to kits which comprise a first composition comprising the one or more anti-CD38 antibodies, and a second composition comprising one or more carfilzomib compounds. In some embodiments, the first and second composition may be mixed together before administering to a subject. In some embodiments, the first and second compositions, may be administered either simultaneously or sequentially (i.e., spaced out over a period of time) so as to obtain the maximum efficacy, additivity, synergy, or a combination thereof of the combination. In some embodiments, the present invention is directed to kits comprising at least one anti-CD38 antibody packaged together with a label having one or more messages that the anti-CD38 antibody shall or might be administered in combination with carfilzomib and optionally with dexamethasone. The kits according to the present invention may further comprise one or more messages that the antibody shall or might be administered to a subject suffering from a blood cancer such as multiple myeloma (e.g., relapsed or refractory multiple myeloma). In some embodiments, the one or more anti-CD38 antibodies in the kits of the present invention are those which are capable of killing a CD38⁺ cell by apoptosis, antibody-dependent cell-mediated cytotoxicity (ADCC), and complement-dependent cytotoxicity (CDC).

In some embodiments, the compositions of the present invention are pharmaceutical compositions. As used herein, the term “pharmaceutical composition” refers to a composition comprising at least one active principle (e.g., an anti-CD38 antibody or a carfilzomib compound) and at least one pharmaceutically acceptable carrier. Pharmaceutically acceptable carriers are well known to the skilled in the art, and usually depend on the chosen route of administration. Pharmaceutical compositions according to the present invention may be provided in any form or formulation that is suitable for the chosen route of administration, such as e.g., a solution in case of an intravenous route of administration, e.g., capsules, pills or tablets in case of an oral route of administration, etc.

The dosage regimen of the active principles and of the pharmaceutical composition described herein can be chosen by prescribing physicians, based on their knowledge of the art, including information published by regulatory authorities. For example, carfilzomib is typically administered intravenously. According to the U.S. Food and Drug Administration (FDA), carfilzomib might be administered intravenously, e.g., over 2 to 10 minutes, on two consecutive days each week for three weeks (Days 1, 2, 8, 9, 15, and 16), followed by a 12-day rest period (Days 17 to 28). In some embodiments, the recommended Cycle 1 dose is 20 mg/m²/day and, if tolerated, the doses of Cycle 2 and subsequent cycles are increased to 27 mg/m²/day. In some embodiments, patients are hydrated prior to and/or following administration. Since, however, co-administration of the one or more anti-CD38 antibodies and the one or more carfilzomib compounds results in an additive or a synergistic effect, the dosing of the carfilzomib compound may be adjusted accordingly, e.g., the dose changed and/or the dosing schedule modified. Of course, prescribing physicians might reconsider which dose and schedule to use depending on the condition and disease status of the patient and based upon clinical and laboratory findings.

As the FDA recommends pre-medication with dexamethasone prior to all Cycle 1 doses, during the first cycle of dose escalation, and if infusion reaction symptoms develop or reappear, the methods and compositions of the present invention may further include dexamethasone, which is member of the glucocorticoid class of steroid drugs, and acts as an anti-inflammatory and immunosuppressant. Thus, in some embodiments, the treatment methods of the present invention further comprise administering a dexamethasone compound to the subject being treated with the one or more anti-CD38 antibodies and the one or more carfilzomib compounds. Similarly, the compositions and kits of the present invention which comprise the one or more anti-CD38 antibodies and/or the one or more carfilzomib compounds may further comprise a dexamethasone compound. As used herein, a “dexamethasone compound” refers to dexamethasone ((8S, 9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one) and dexamethasone derivatives. As used herein, a “dexamethasone derivative” refers to a compound having the following structural formula:

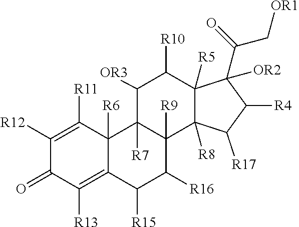

wherein R1-R17 are each independently H, a halogen, an alkyl, an alkoxy, amino, or an alkylamine. In some preferred embodiments, R1-R3 are H. In some preferred embodiments, R4-R6 are methyl. In some preferred embodiments, R7 is a halogen, preferably fluorine. In some preferred embodiments, R8 is H. In some preferred embodiments, R1-R3 are H, R4-R6 are methyl, R7 is a halogen, preferably fluorine, and R8 is H.

In some embodiments, the dexamethasone compound may be administered orally. In some embodiments, the dexamethasone compound may be administered at the same or a lower dose than the dose recommended for dexamethasone by the EMA.

The compositions of the present invention may be used as a medicament and/or for use in the manufacture of a medicament. In some embodiments, the compositions of the present invention may be used as a medicament and/or for use in the manufacture of a medicament for use in the treatment of a cancer such as a hematological malignancy of the blood, bone marrow, and/or lymph nodes, preferably a blood cancer.

Several documents are cited throughout the text of this specification. Each of the documents herein (including any journal article or abstract, published or unpublished patent application, issued patent, manufacturer's specifications, instructions, etc.) are hereby incorporated by reference. However, there is no admission that any document cited herein is indeed prior art in respect of the present invention.

The following examples are intended to illustrate but not to limit the invention.

### EXAMPLES

hu38SB19 was provided in solution at 5 mg/ml, stored at 4° C. It was diluted into sterile saline in preparation for dosing, stored at 4° C. and used within 10 days of dilution.

Carfilzomib (PR-171) was obtained from Chemie Tek (CT-CARF 98). Carfilzomib was formulated in an aqueous solution of 10% (w/v) sulfobutylether-h-cyclodextrin (Cydex) and 10 mmol/L sodium citrate (pH 3.5), 2 mg/ml stock prepared and frozen at −80° C., diluted daily with vehicle before injection. Carfilzomib was administered weekly qdx2×3 wk (iv).

### Example 1: Effect of the Administration of Both Anti-CD38 Antibody and Carfilzomib in a Mice Model of MM

These studies under this Example were done under approval of the UCSF IACUC.

The subcutaneous multiple myeloma (MM) xenograft mouse models were established using NCI-H929 or RPMI-8226 cell lines. Specifically, 5-6 week old female Balb/c Scid mice were obtained from Jackson Lab. Mice were housed for 7-10 days prior to implantation. Mice were housed in a dedicated room in the UCSF Mt Zion Animal Barrier Facility. NCI-H929 and RPMI-8226 cells were obtained from the German Collection of Microorganisms and Cell Cultures, DSMZ, (Deutsche Sammlung von Mikroorganismen und Zellkulturen), and grown in sterile suspension culture in T225 flasks as follows: NCI-H929: RPMI1640+20% FBS+4 mM L-glutamine+1 mM sodium pyruvate+50 μM mercaptoethanol. RPMI-8226: RPMI1640+10% FBS+4 mM L-glutamine.

At the time of implantation, mice were shaved on the right flank and shoulder region and anesthetized with ip avertin. MM cells suspended in serum free RPMI 1640 media diluted 1:1 with Matrigel (BD) at a concentration of 1×10⁸ cells per ml were injected sc into the right flank in 100 μL volume (1×10⁷ cells) using a 1 ml syringe and 25 g needle. Mice were monitored twice weekly for the appearance of tumors and once tumors were visible, measurements were collected twice weekly for body weight and tumor volume. Electronic balance and calipers were used and data was collected directly into a study management program (Study Director). When the mean tumor volume reached about 150-200 mm³, the mice were distributed into treatment groups of 8-10 mice per groups and dosing was begun.

The dosing schedule was hu38SB19 was 2×/wk×2 wk (iv. lateral tail vein) and carfilzomib was weekly qdx2×3 wk (iv, lateral tail vein) (once per day, two days a week for three weeks). Dose levels for use in combination studies are as follows:

| Cell Type | Carfilzomib | hu38SB19 |
|---|---|---|
| NCI-H929 | 2 mpk | 0.5 mpk |
| RPMI 8226 | 2.5 mpk | 15 mpk |

mpk = mg per kg body weight

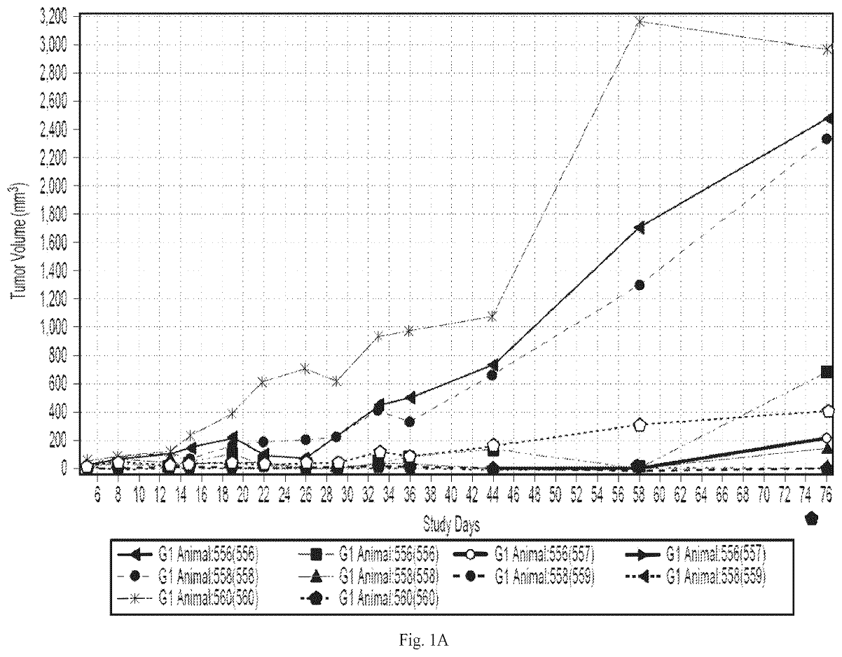

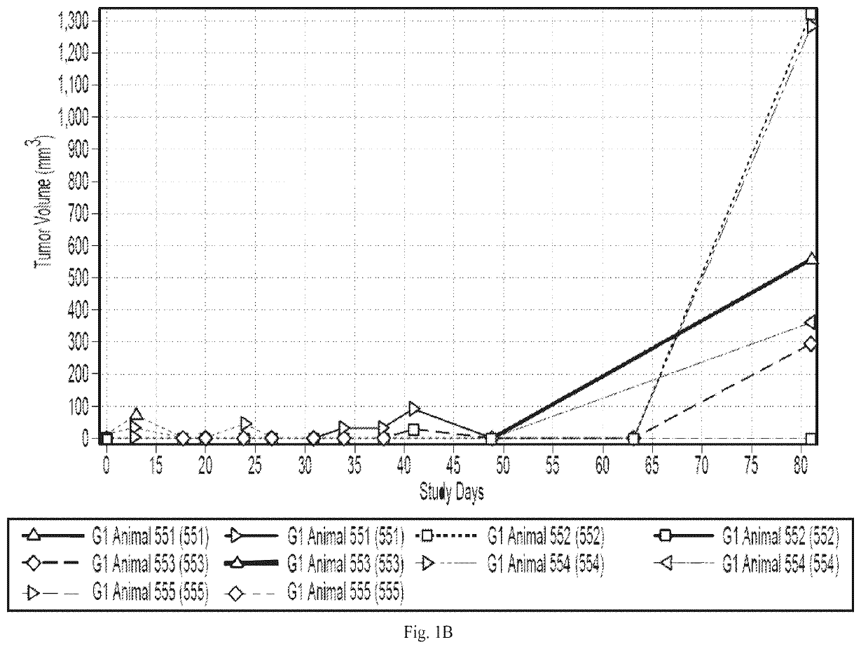

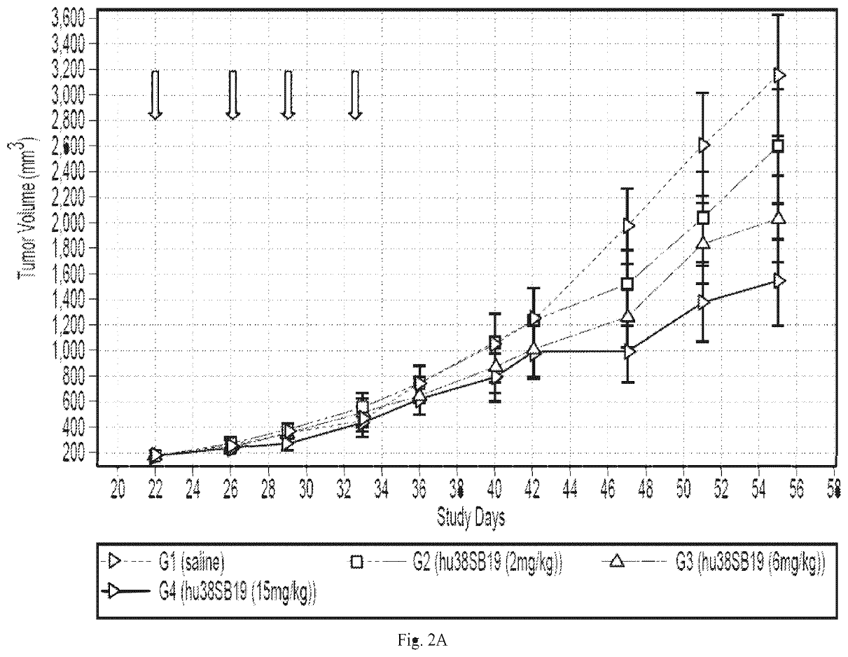

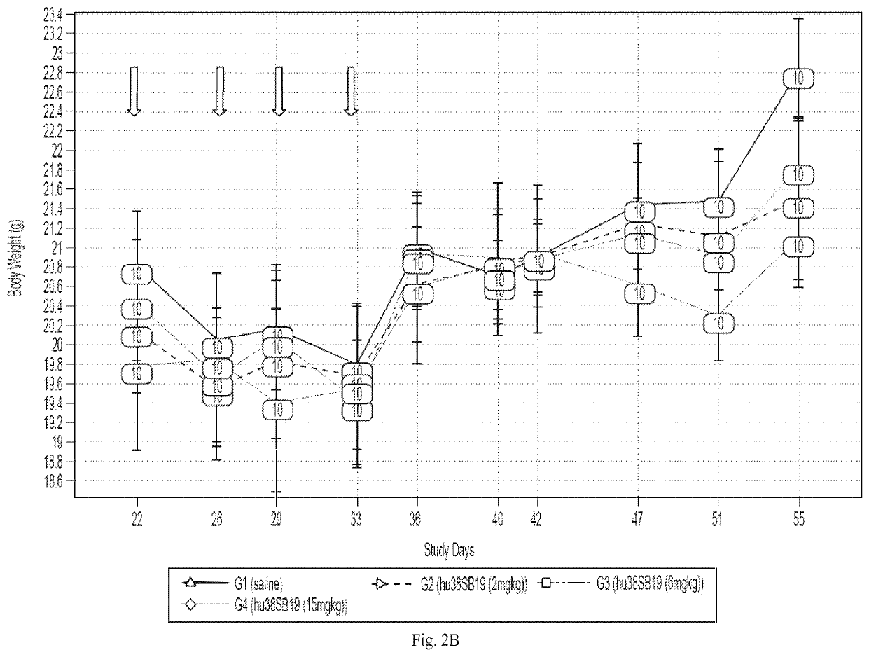

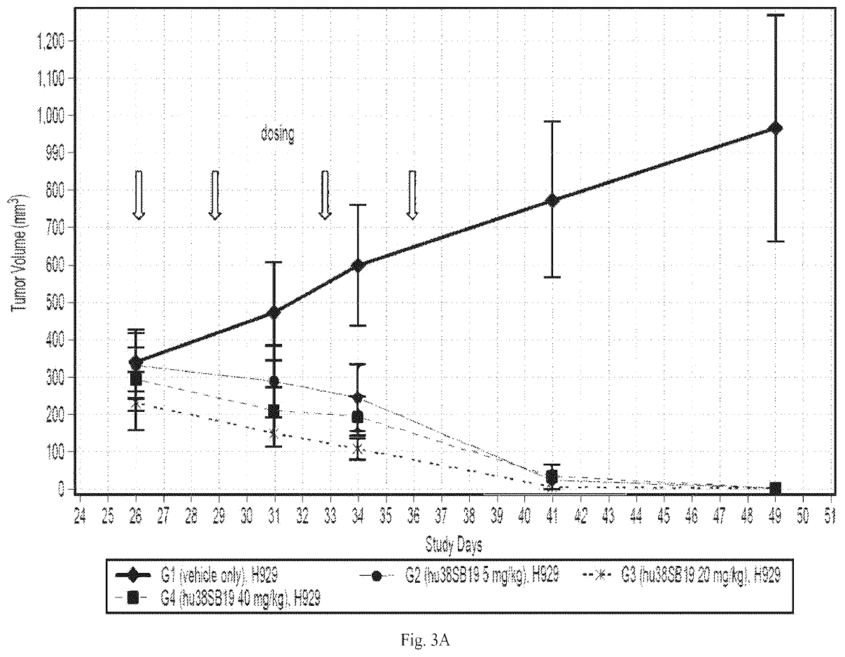

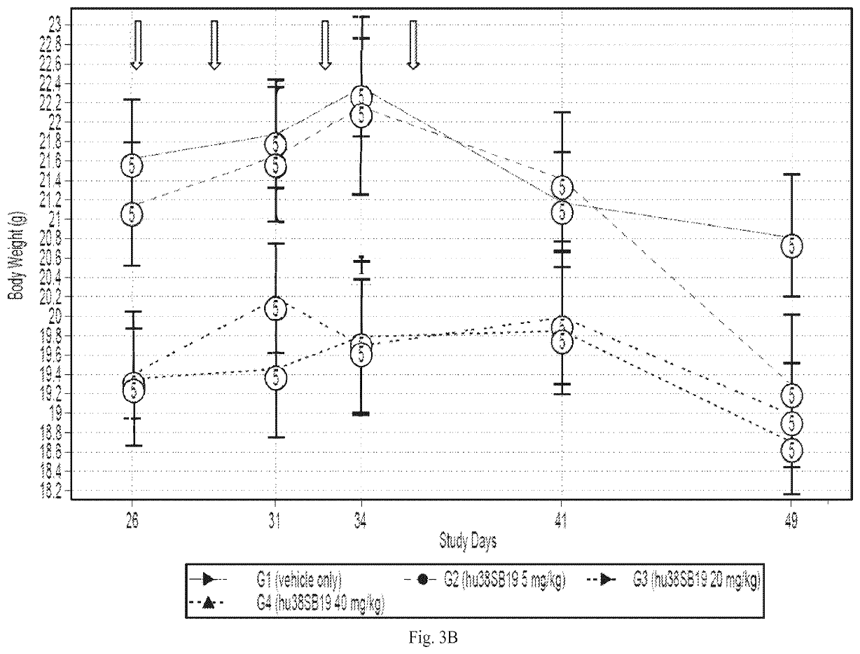

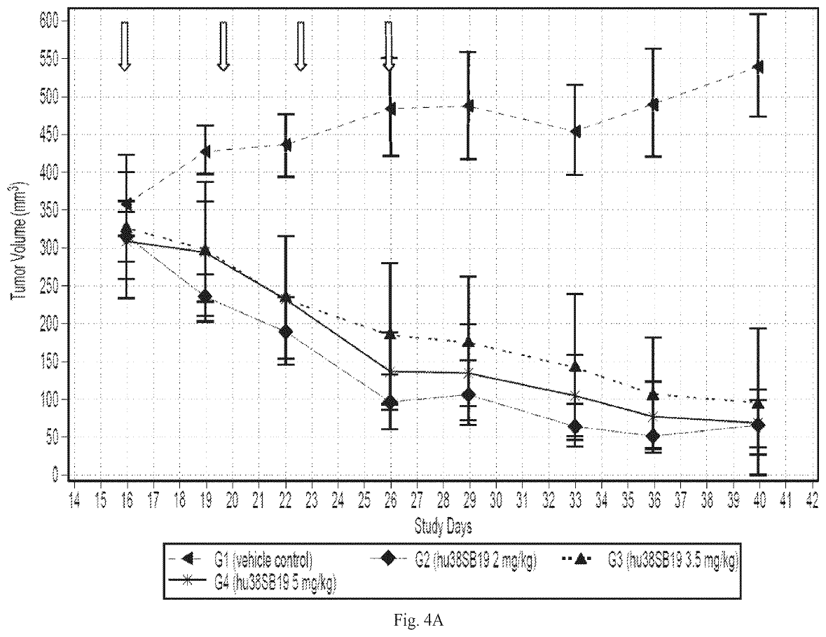

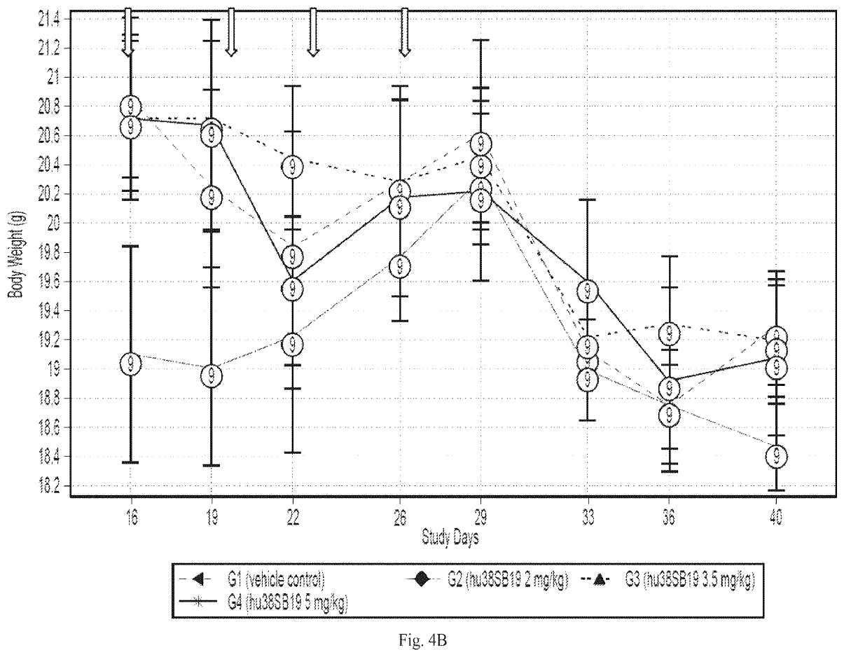

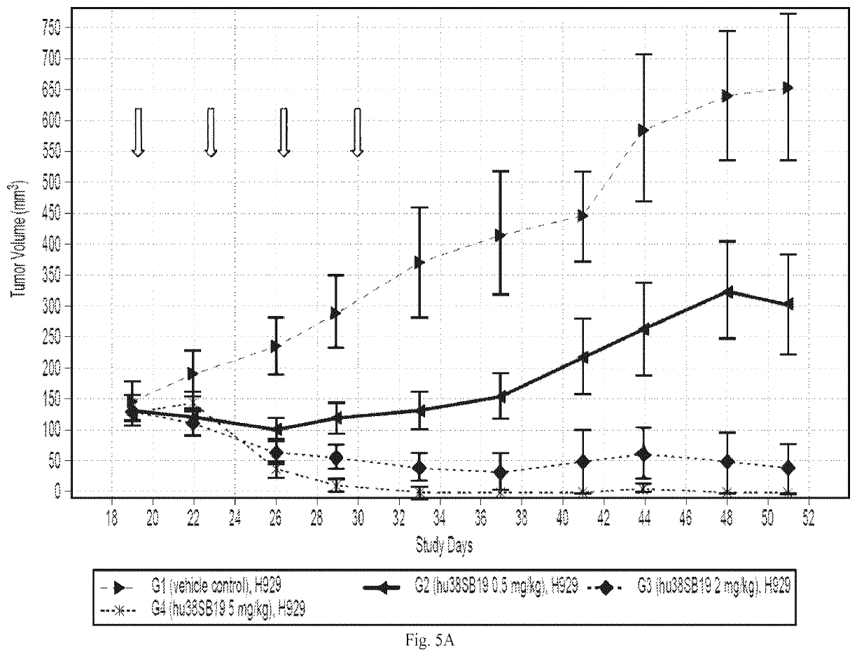

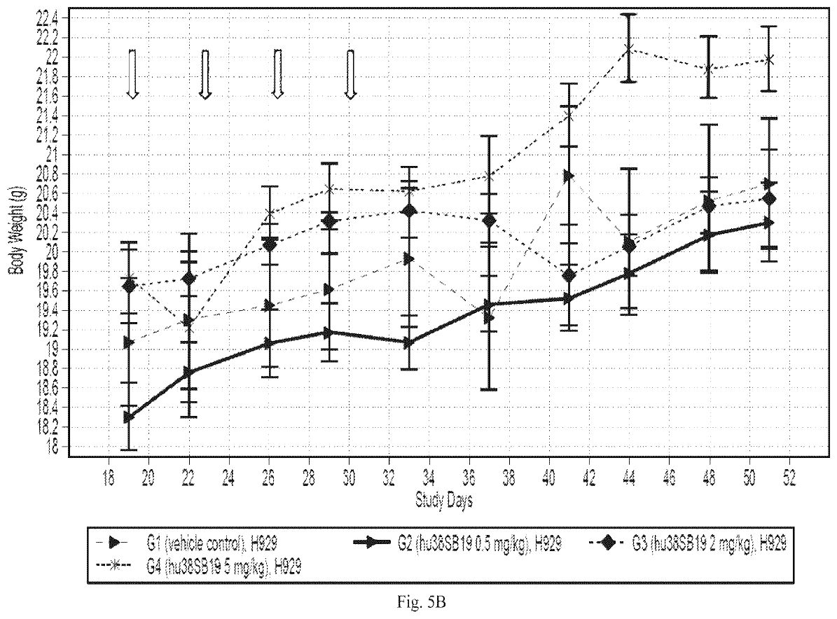

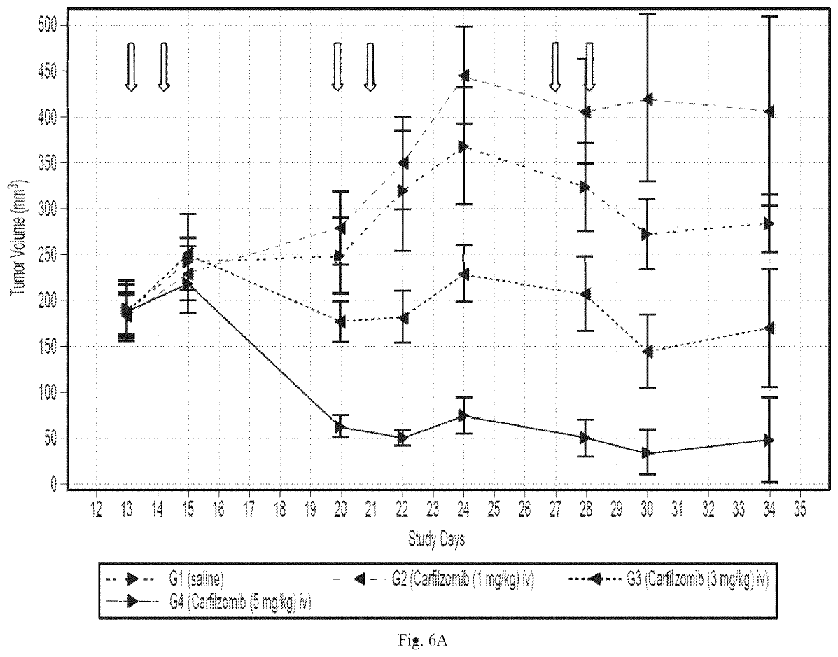

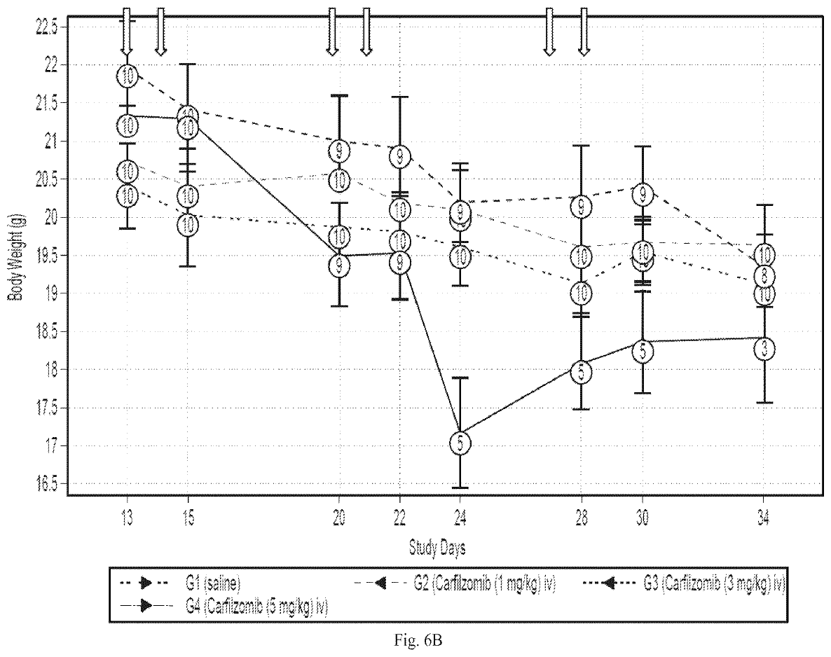

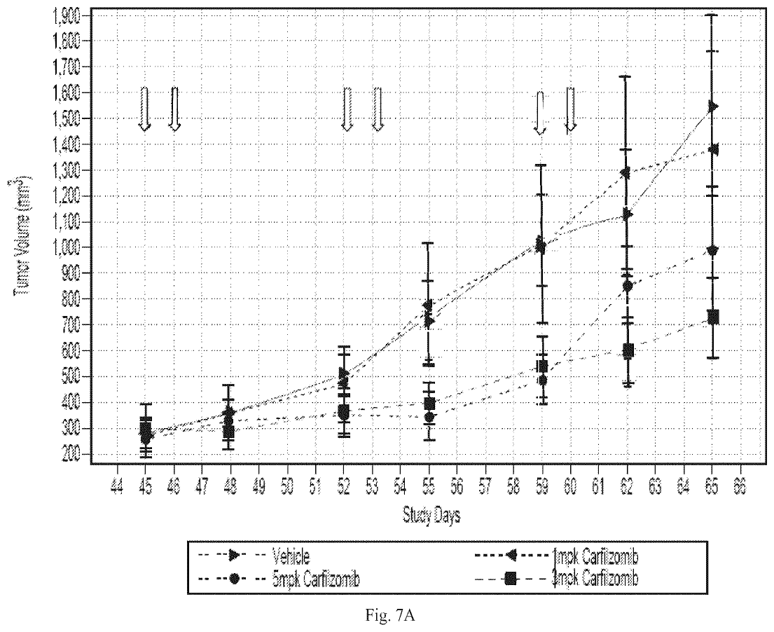

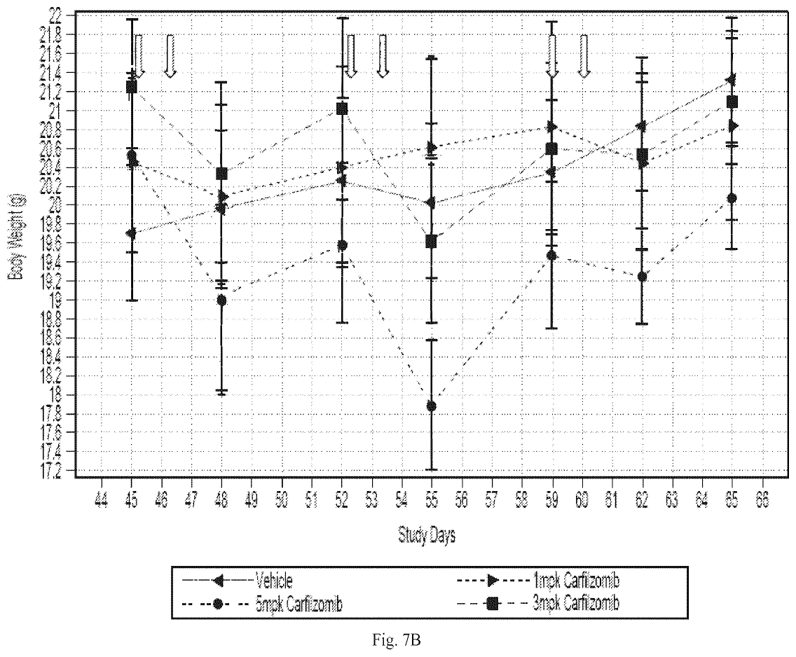

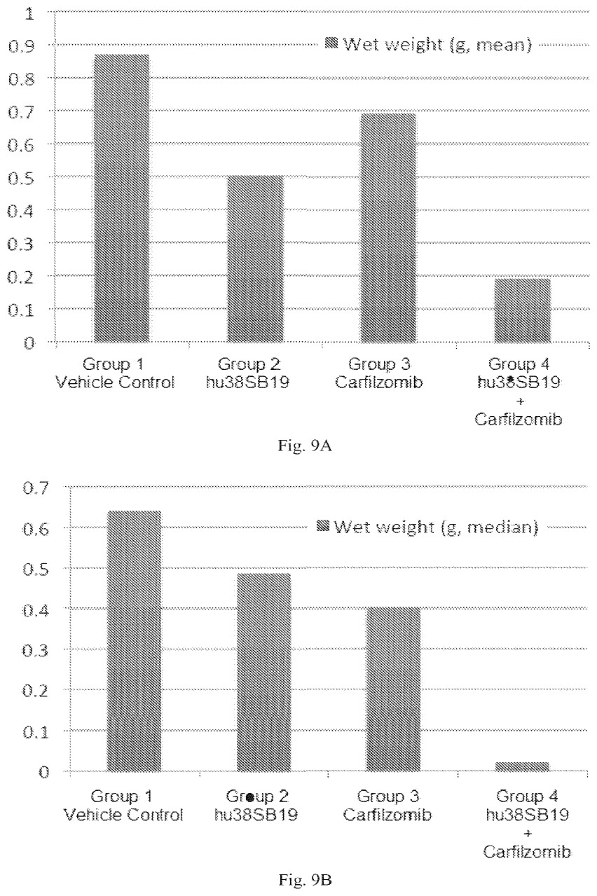

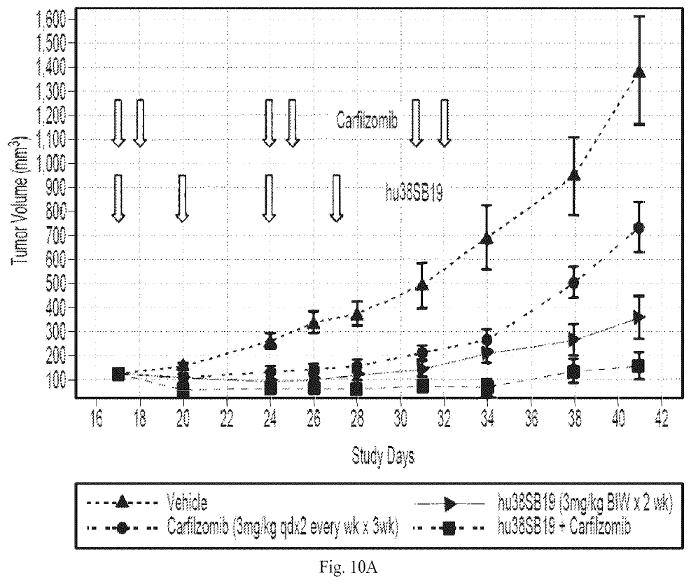

Data were collected using electronic balance and calipers using a study management application called StudyLog (Study Director). Graphs are taken directly from the application. The experimental results are provided in FIGS. 1A-10B.

Based on the single agent results of hu38SB19 and carfilzomib in RPMI-8226 and NCI-H929 multiple myeloma xenograft models, the H929 model appears to be a more sensitive model to both agents while the RPMI model seems to be more resistant to the treatments even at the highest doses tested (FIGS. 1A-7B). Therefore in the combination studies, a suboptimal dose for each agent was chosen to evaluate the activity of the combination treatment (carfilzomib hu38SB19) in the H929 model while higher doses of carfilzomib and hu38SB19 were tested in the RPMI model.

Antitumor activity was determined according to NCI standards based on the ratio of the median tumor volume change of the treated/median tumor volume change of the control×100 (% ΔT/ΔC). Low numerical values for ΔT/ΔC describe stronger anti-tumor activity. Anti-tumor activity is defined as T/C≤40% at minimum. ΔT/ΔC<10% is considered high anti-tumor activity.

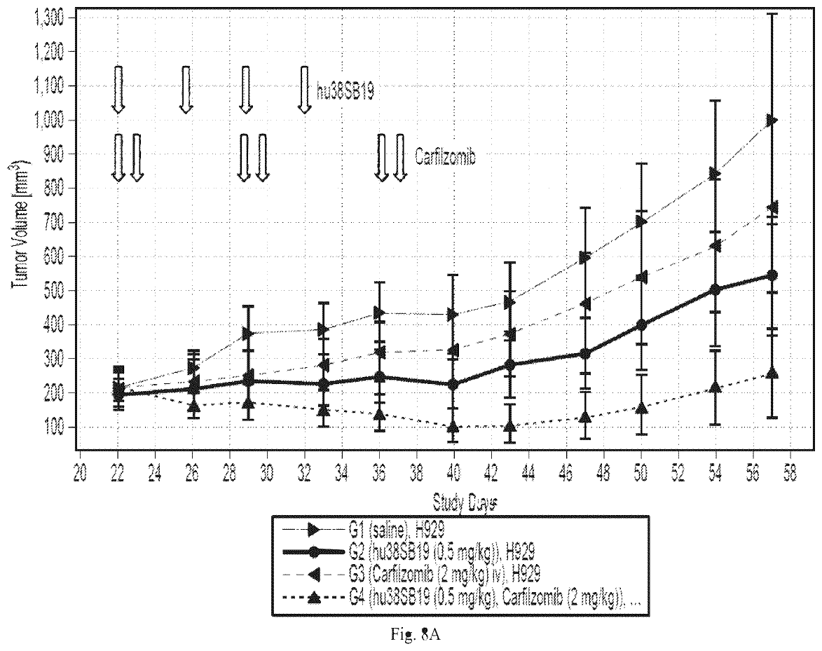

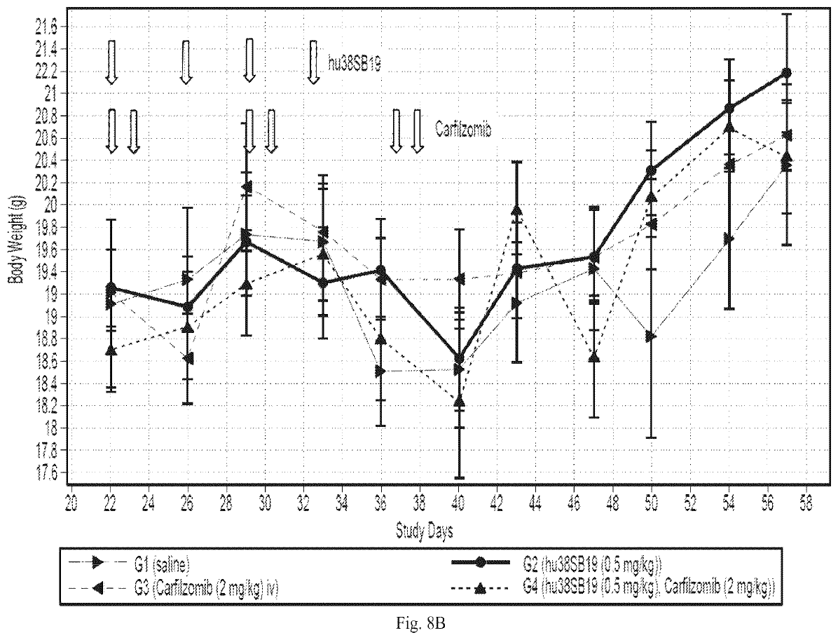

In the H929 model, hu38SB19 alone at 0.5 mg/kg/injection (twice a week for 2 weeks) was inactive with a % ΔT/ΔC of 74%. Treatment with carfilzomib alone at 2 mg/kg (twice a week for three weeks) was inactive (68% ΔTΔ/C). The combination of hu38SB19 (0.5 mg/kg/injection) and carfilzomib (2 mg/kg/injection) had much higher activity (tumor regression) with % ΔT/ΔC of-11% (FIG. 8). The results are summarized in Table 1.

TABLE 1
Anti-tumor efficacy of hu38SB19 in combination with
carfilzomib against NCI-H929 multiple myeloma model

| | Dose in | Schedule of | | |
|---|---|---|---|---|
| | mg/kg | Administration | % ΔT/ΔC | |
| Agent | (total dose) | IV route | (D69) | Activity |
| PBS | — | 2×/wk × 2 wk (IV) | | |
| hu38SB19 | 0.5 (2) | 2×/wk × 2 wk (IV) | 74 | Inactive |
| Carfilzomib | 2 (12) | 2×/wk × 3 wk (IV) | 68 | Inactive |
| hu38SB19 + | 0.5 (2) + | 2×/wk × 2 wk (IV) + | −11 | Highly |
| Carfilzomib | 2 (12) | 2×/wk × 3 wk (IV) | | Active |

% ΔT/ΔC Median tumor volume change of the treated/Median tumor volume change of the control × 100, IV = intravenous, wk = week, PBS: phosphate buffered saline

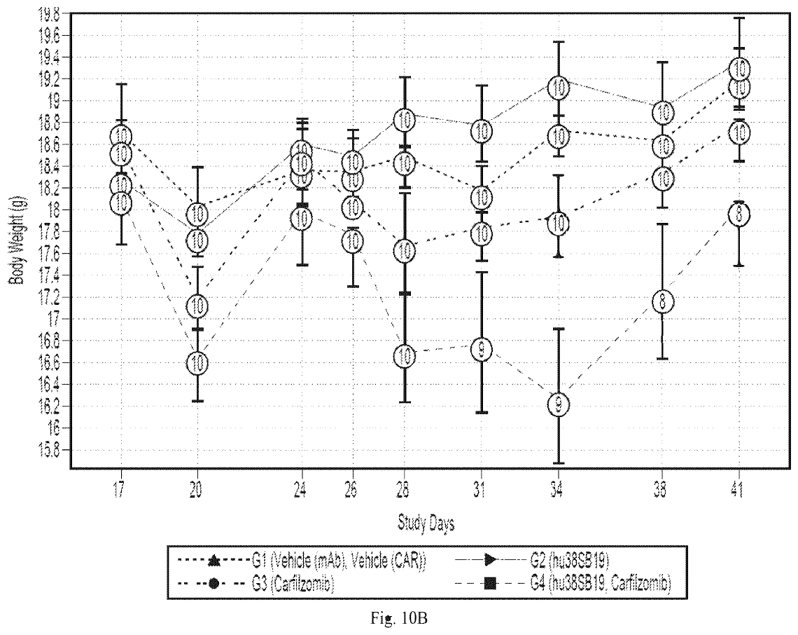

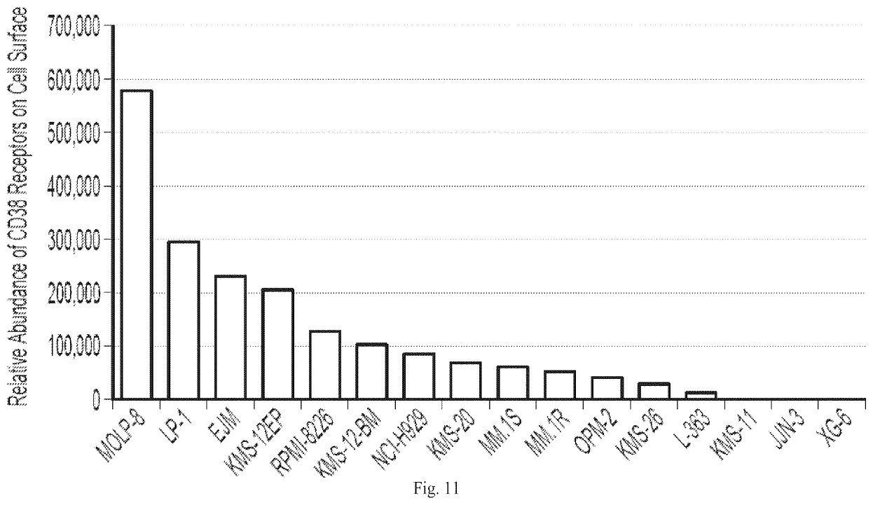

As shown in FIGS. 10A-10B, similar results were obtained in the RPMI-8226 xenograft models. In particular, on Day 41, carfilzomib (3 mg/kg qdx2 every wk×3 wk) resulted in 0/10 complete regressions; hu38SB19 (3 mg/kg BIW×2 wk) resulted in 2/10 complete regressions. Thus, the additive expectation based on extrapolation for the combination of carfilzomib and hu38SB19 would be expected to be 2/10 complete regressions. However, the combination of carfilzomib and hu38SB19 surprisingly resulted in 5/8 complete regressions which is more than 3 times the expected result.

In both the NCI-H929 and RPMI-8226 xenograft models, the combination treatment inhibited tumor growth to a much greater extent than a single agent alone, indicating the combination of hu38SB19 and carfilzomib blocked tumor cell growth through potential synergistic mechanisms. Carfilzomib is a second generation proteasome inhibitor which was recently approved to treat relapsed and refractory multiple myeloma patients. Inhibition of proteasome activity by carfilzomib results in a build-up of polyubiquinated proteins, which may cause cell cycle arrest, apoptosis, and inhibition of tumor growth. Hu38SB19 has demonstrated multiple mechanisms of action including ADCC, CDC, and direct apoptosis induction.

It has been reported that some CD38 antibodies such as Daratumumab is able to induce apoptosis only after cross-linking with a secondary antibody without much direct effect by itself. However, in preclinical studies, hu38SB19 demonstrated potent direct pro-apoptotic activity on tumor cells without cross-linking. Thus, this unique property of hu38SB19 may also lead to greater tumor cell killing when in combination with carfilzomib compared to other CD38 antibodies combined with carfilzomib.

### Example 2: Effect of the Administration of Both Anti-CD38 Antibody and Carfilzomib in Humans

A clinical study for evaluating the effects of a treatment with hu38SB19 combined with carfilzomib in patients with relapsed or refractory multiple myeloma may be performed as described below.

The goals of the study may include:
To determine the efficacy and the maximum tolerated dose;
To evaluate the safety, including immunogenicity, of hu38SB19 in combination with carfilzomib in relapse or refractory multiple myeloma. The severity, frequency and incidence of all toxicities is assessed;
To evaluate the pharmacokinetics (PK) of hu38SB19 when administered in combination with carfilzomib and the PK of carfilzomib in combination with hu38SB19, and optionally dexamethasone.
To assess the relationship between clinical (adverse event and/or tumor response) effects and pharmacologic parameters (PK/pharmacodynamics), and/or biologic (correlative laboratory) results;
Estimate the activity (response rate) using International Myeloma Working Group defined response criteria of hu38SB19 plus carfilzomib, and optionally dexamethasone; and
To describe overall survival, progression free survival (PFS) and time to disease progression in patients treated with this combination.

Patients with relapsed multiple myeloma who have received at least two prior treatments (including bortezomib and thalidomide and/or lenalidomide) and whose disease has a less than or equal to 25% response to the most recent therapy or has disease progression during or within 60 days of the most recent therapy are enrolled. Patients excluded from the trial are those having total bilirubin levels ≥2×upper limit of normal (ULN); creatinine clearance rates <30 mL/min; New York Heart Association Class III to IV congestive heart failure; symptomatic cardiac ischemia; myocardial infarction within the last 6 months; peripheral neuropathy Grade 3 or 4, or peripheral neuropathy Grade 2 with pain; active infections requiring treatment; and pleural effusion.

Carfilzomib is administered intravenously over 2 to 0.10 minutes on two consecutive days each week for three weeks, followed by a 12-day rest period (28-day treatment cycle), until disease progression, unacceptable toxicity, or for a maximum of 12 cycles. Patients receive 20 mg/m² at each dose in Cycle 1, and 27 mg/m² in subsequent cycles. To reduce the incidence and severity of fever, rigors, chills, dyspnea, myalgia, and arthralgia, dexamethasone 4 mg by mouth or by intravenous infusion may be administered prior to all carfilzomib doses during the first cycle and prior to all carfilzomib doses during the first dose-escalation (27 mg/m2) cycle. Dexamethasone premedication (4 mg orally or intravenously) may be reinstated if these symptoms reappeared during subsequent cycles. Doses of hu38SB19 may be administered on the same days the carfilzomib doses are administered and/or on different days. When administered on the same days, hu38SB19 and carfilzomib may be administered at the same time as one composition or as two separate compositions.

The study duration for an individual patient includes a screening period for inclusion of up to 21 days, and at least 4 weeks of treatment in the absence of severe adverse reaction, dose limiting toxicity or disease progression plus up to 60 days post-treatment follow up. The total duration of the study may be up to one year.

The following parameters may be measured during and/or at the end of the study:
Number of patients with adverse events when treated with hu38SB19 in combination with carfilzomib;
Assessment of partial response, complete response, progression free survival, and survival;
Assessment of the following PK parameters: area under curve (AUC), maximum concentration (Cmax) and plasma half-life (T ½);
Number of CD38 receptors occupied by hu38SB19; and
Number of anti-SAR antibodies in response to hu38SB19.

### Example 3: Efficacy of Anti-CD38 Antibody in In Vivo Tumor Models of Multiple Myeloma as a Single-Agent or in Combination with and Carfilzomib

### A. Materials and Methods

CD38 Density: CD38 density was determined using anti-CD38-PE Quantibrite (BD Biosciences; Cat. 342371) per the manufacturer's recommended protocols.

Reagents & Compounds: hu38SB19 was provided by Sanofi Oncology in solution at 5 mg/ml and stored at 4° C. hu38SB19 was diluted into sterile saline in preparation for dosing and used within 10 days of dilution. hu38SB19 was administered twice weekly ×2 wk IV. Carfilzomib (PR-171) was obtained from Chemie Tek (CT-CARF 98). Carfilzomib was formulated in an aqueous solution of 10% (w/v) sulfobutylether-h-cyclodextrin (Cydex) and 10 mmol/L sodium citrate (pH 3.5), 2 mg/ml stock prepared and frozen at −80° C., diluted daily with vehicle before injection. Carfilzomib was administered weekly qdx2×3 wk (iv).

Test Animals: 5-6 week old female Balb/c Scid mice were obtained from Jackson Lab. Mice were housed for 7-10 days prior to implantation of multiple myeloma (MM) cell lines. Mice were housed in a dedicated room in the UCSF Mt. Zion Animal Barrier Facility.

Xenograft Model: At the time of implantation, mice were shaved on the right flank and shoulder. MM cells were suspended in serum free RPMI 1640 media diluted 1:1 with Matrigel (BD) at a concentration of 1×10⁸ cells per ml were injected sc into the right flank in 100 ul volume (1×10⁷ cells) using a 1 ml syringe and 25 g needle. Mice were monitored twice weekly for the appearance of tumors and once tumors were visible, measurements were collected twice weekly for body weight and tumor volume. Electronic balance and calipers were used and data was collected directly into a study management program (Study Director). When the mean tumor volume reached approximately 150-200 mm³, mice were distributed into treatment groups of 8-10 mice per group and dosing was initiated.

### B. Summary and Conclusions

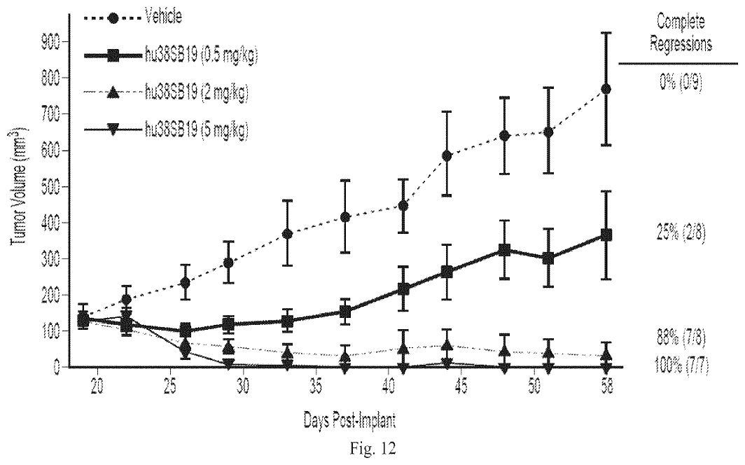

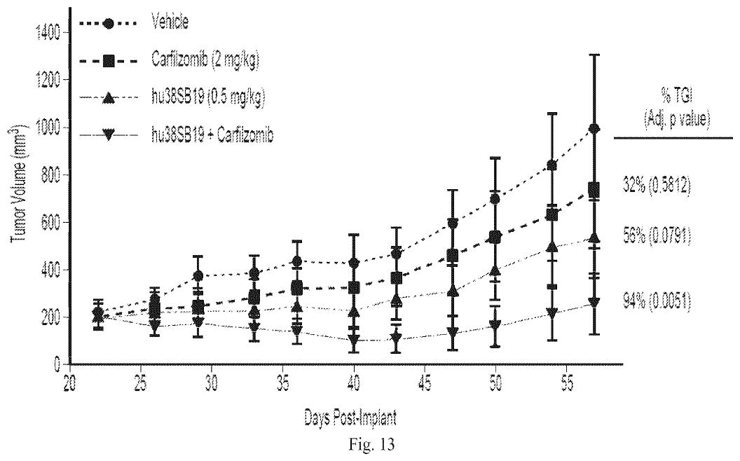

hu38SB19 is a humanized anti-CD38 antibody whose anti-myeloma effects incorporate mechanisms of ADCC, CDC, and direct apoptosis. FIG. 11 shows the cell surface density of CD38 in multiple myeloma cell lines. See Kim D, Park C Y, Medeiros B C, Weissman IL. CD19-CD45 low/-CD38 high/CD138+ plasma cells enrich for human tumorigenic myeloma cells. Leukemia. 2012 Dec. 26 (12): 2530-7. CD38-positive multiple myeloma plasma cells demonstrate variable CD38 cell surface densities. All cell lines, with the exception of XG-6, are reported as CD38-positive. See Bataille R, Jégo G, Robillard N, et al. The phenotype of normal, reactive and malignant plasma cells. Identification of “many and multiple myelomas” and of new targets for myeloma therapy. Haematologica. 2006 September, 91(9):1234-40. Binding of hu38SB19 to CD38 also impinges on the ADPRC enzymatic activity of CD38. In vivo, hu38SB19 demonstrates potent anti-tumor effects in multiple myeloma xenografts, a disease largely characterized by neoplastic plasma cells expressing CD38. FIG. 12 shows that single-agent administration of hu38SB19 results in dose-dependent inhibition of tumor growth in an NCI-H929 hind-flank model. The magnitude and significance of tumor growth inhibition at the end of the study increased with increased doses of hu38SB19. FIG. 13 shows that a combined regimen of hu38SB19 and carfilzomib results in significant tumor growth inhibition in an NCI-H929 xenograft model that is not robustly sensitive to single-agent therapy with carfilzomib. These data demonstrate that single-agent hu38SB19 inhibits growth of NCI-H929 tumors and combines with sub-efficacious doses of carfilzomib to produce significant inhibition of tumor growth. Taken together, these data support further evaluation of hu38SB19, both as a single-agent and in combination with standard-of-care treatment regimens, as a potential therapy for the treatment of multiple myeloma.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1: A method of treating a cancer in a subject, comprising administering one or more anti-CD38 antibodies and one or more carfilzomib compounds to the subject.

2: The method of claim 1, wherein the cancer is a hematological malignancy.

3: The method of claim 1, wherein the cancer is multiple myeloma.

4: The method of claim 1, wherein the cancer is a relapsed multiple myeloma or a refractory multiple myeloma.

5: The method of claim 1, wherein the one or more carfilzomib compounds is carfilzomib.

6: The method of claim 1, wherein the one or more anti-CD38 antibodies are administered in an effective amount, preferably a synergistic amount.

7: The method of claim 1, wherein the one or more anti-CD38 antibodies and/or the one or more carfilzomib compounds are administered in a therapeutically effective amount.

8: The method of claim 1, wherein at least one of the one or more anti-CD38 antibodies is capable of killing a CD38⁺ cell by apoptosis, antibody-dependent cell-mediated cytotoxicity (ADCC), and complement-dependent cytotoxicity (CDC).

9: The method of claim 1, wherein at least one of the one or more anti-CD38 antibodies comprises one or more complementarity-determining region having an amino acid sequence selected from the group consisting of SEQ ID NOs: 13, 14, 81, 15, 16, 17, 18, 1, 2, 3, 4, 5, 6, 7, 8, 9, 10, 11, 12, 19, 20, 21, 22, 23, 24, 25, 26, 27, 28, 29, 30, 31, 32, 33, 34, 35 and 36.

10: The method of claim 1, wherein at least one of the one or more anti-CD38 antibodies is selected from the group consisting of:
a) an antibody comprising a heavy chain comprising three sequential CDRs having amino acid sequences consisting of SEQ ID NOs: 13, 15 and either SEQ ID NO: 14 or SEQ ID NO: 81, and a light chain comprising three sequential CDRs having amino acid sequences consisting of SEQ ID NOs: 16, 17 and 18;
b) an antibody comprising a heavy chain comprising three sequential CDRs having amino acid sequences consisting of SEQ ID NOs: 25, 26 and 27, and a light chain comprising three sequential CDRs having amino acid sequences consisting of SEQ ID NOs: 28, 29 and 30;
c) an antibody comprising a heavy chain comprising three sequential CDRs having amino acid sequences consisting of SEQ ID NOs: 1, 2 and 3, and a light chain comprising three sequential CDRs having amino acid sequences consisting of SEQ ID NOs: 4, 5 and 6;
d) an antibody comprising a heavy chain comprising three sequential CDRs having amino acid sequences consisting of SEQ ID NOs: 7, 8 and 9, and a light chain comprising three sequential CDRs having amino acid sequences consisting of SEQ ID NOs: 10, 11 and 12;
e) an antibody comprising a heavy chain comprising three sequential CDRs having amino acid sequences consisting of SEQ ID NOs: 19, 20 and 21, and a light chain comprising three sequential CDRs having amino acid sequences consisting of SEQ ID NOs: 22, 23 and 24; and
f) an antibody comprising a heavy chain comprising three sequential CDRs having amino acid sequences consisting of SEQ ID NOs: 31, 32 and 33, and a light chain comprising three sequential CDRs having amino acid sequences consisting of SEQ ID NOs: 34, 35 and 36.

11-26. (canceled)


SEQUENCE LISTING (81 sequences)

SEQ ID NO: 1 - protein, 5 aa, Mus sp.
SYGMN

SEQ ID NO: 2 - protein, 17 aa, Mus sp.
WINTYTGEPTYADDFKG

SEQ ID NO: 3 - protein, 5 aa, Mus sp.
RGFAY

SEQ ID NO: 4 - protein, 15 aa, Mus sp.
RASESVEIYGNGFMN

SEQ ID NO: 5 - protein, 7 aa, Mus sp.
RASNLES

SEQ ID NO: 6 - protein, 9 aa, Mus sp.
QQINEDPFT

SEQ ID NO: 7 - protein, 5 aa, Mus sp.
NSGMN

SEQ ID NO: 8 - protein, 17 aa, Mus sp.
WINTYTGEPTYADDFKG

SEQ ID NO: 9 - protein, 5 aa, Mus sp.
RGFVY

SEQ ID NO: 10 - protein, 15 aa, Mus sp.
RASESVAIYGNSFLK

SEQ ID NO: 11 - protein, 7 aa, Mus sp.
RASNLES

SEQ ID NO: 12 - protein, 9 aa, Mus sp.
QQINEDPYT

SEQ ID NO: 13 - protein, 5 aa, Mus sp.
DYWMQ

SEQ ID NO: 14 - protein, 17 aa, Mus sp.
TIYPGDGDTGYAQKFKG

SEQ ID NO: 15 - protein, 11 aa, Mus sp.
GDYYGSNSLDY

SEQ ID NO: 16 - protein, 11 aa, Mus sp.
KASQDVSTVVA

SEQ ID NO: 17 - protein, 7 aa, Mus sp.
SASYRYI

SEQ ID NO: 18 - protein, 9 aa, Mus sp.
QQHYSPPYT

SEQ ID NO: 19 - protein, 5 aa, Mus sp.
GSWMN

SEQ ID NO: 20 - protein, 17 aa, Mus sp.
RIYPGDGDIIYNGNFRD

SEQ ID NO: 21 - protein, 10 aa, Mus sp.
WGTFTPSFDY

SEQ ID NO: 22 - protein, 11 aa, Mus sp.
KASQDVVTAVA

SEQ ID NO: 23 - protein, 7 aa, Mus sp.
SASHRYT

SEQ ID NO: 24 - protein, 9 aa, Mus sp.
QQHYTTPTT

SEQ ID NO: 25 - protein, 5 aa, Mus sp.
SYTLS

SEQ ID NO: 26 - protein, 17 aa, Mus sp.
TISIGGRYTYYPDSVEG

SEQ ID NO: 27 - protein, 8 aa, Mus sp.
DFNGYSDF

SEQ ID NO: 28 - protein, 11 aa, Mus sp.
KASQVVGSAVA

SEQ ID NO: 29 - protein, 7 aa, Mus sp.
WASTRHT

SEQ ID NO: 30 - protein, 9 aa, Mus sp.
QQYNSYPYT

SEQ ID NO: 31 - protein, 5 aa, Mus sp.
NFGMH

SEQ ID NO: 32 - protein, 17 aa, Mus sp.
YIRSGSGTIYYSDTVKG

SEQ ID NO: 33 - protein, 11 aa, Mus sp.
SYYDFGAWFAY

SEQ ID NO: 34 - protein, 11 aa, Mus sp.
KASQNVGTNVA

SEQ ID NO: 35 - protein, 7 aa, Mus sp.
SASSRYS

SEQ ID NO: 36 - protein, 9 aa, Mus sp.
QQYNSYPLT

SEQ ID NO: 37 - DNA, 336 bp, Mus sp.
  source 1..336: mol_type = unassigned DNA
  CDS 1..336: protein_id = 38; translation = NIVLTQSPASLAVSLGQRATISCRASESVEIYGNGFMNWFQQKP GQPPKLLIYRASNLESGIPARFSGSGSRTEFTLTIDPVEADDVATYYCQQINEDPFTF GSGTKLEIKR
aacattgtgctgacccaatctccagcttctttggctgtgtctcttgggcagagggccaccatatcctgcagagccagtgaaagtgttgagatttatggcaatggttttatgaactggttccagcagaaaccaggacagccacccaaactcctcatctatcgtgcatccaacctagaatctgggatccctgccaggttcagtggcagtgggtctaggacagagttcaccctcaccattgatcctgtggaggctgatgatgttgcaacctattactgtcaacaaattaatgaggatccattcacgttcggctcggggacaaagttggaaataaaacgg

SEQ ID NO: 38 - protein, 112 aa, Mus sp.
NIVLTQSPASLAVSLGQRATISCRASESVEIYGNGFMNWFQQKPGQPPKLLIYRASNLESGIPARFSGSGSRTEFTLTIDPVEADDVATYYCQQINEDPFTFGSGTKLEIKR

SEQ ID NO: 39 - DNA, 336 bp, Mus sp.
  source 1..336: mol_type = unassigned DNA
  CDS 1..336: protein_id = 40; translation = DIVLTQSPASLAVSLGQRATISCRASESVAIYGNSFLKWFQQKP GQPPKLLIYRASNLESGIPARFSGSGSGTDFTLTINPVEADDVATYYCQQINEDPYTF GGGTKLEIKR
gacattgtactgacccaatctccagcttctttggctgtgtctctagggcagagggccaccatatcctgcagagccagtgagagtgttgctatttatggcaatagttttctgaaatggttccagcagaaaccgggacagccacccaaactcctcatctatcgtgcatccaacctagaatctgggatccctgccaggttcagtggcagtgggtctgggacagacttcaccctcaccattaatcctgtggaggctgatgatgttgcaacctattactgtcagcaaattaatgaggatccgtacacgttcggaggggggaccaagctggaaataaaacgg

SEQ ID NO: 40 - protein, 112 aa, Mus sp.
DIVLTQSPASLAVSLGQRATISCRASESVAIYGNSFLKWFQQKPGQPPKLLIYRASNLESGIPARFSGSGSGTDFTLTINPVEADDVATYYCQQINEDPYTFGGGTKLEIKR

SEQ ID NO: 41 - DNA, 324 bp, Mus sp.
  source 1..324: mol_type = unassigned DNA
  CDS 1..324: protein_id = 42; translation = DIVMAQSHKFMSTSVGDRVSITCKASQDVSTVVAWYQQKPGQSP KRLIYSASYRYIGVPDRFTGSGSGTDFTFTISSVQAEDLAVYYCQQHYSPPYTFGGGT KLEIKR
gacattgtgatggcccagtctcacaaattcatgtccacatcagttggagacagggtcagcatcacctgcaaggccagtcaggatgtgagtactgttgtggcctggtatcaacagaaaccaggacaatctcctaaacgactgatttactcggcatcctatcggtatattggagtccctgatcgcttcactggcagtggatctgggacggatttcactttcaccatcagcagtgtgcaggctgaagacctggcagtttattactgtcagcaacattatagtcctccgtacacgttcggaggggggaccaagctggaaataaaacgg

SEQ ID NO: 42 - protein, 108 aa, Mus sp.
DIVMAQSHKFMSTSVGDRVSITCKASQDVSTVVAWYQQKPGQSPKRLIYSASYRYIGVPDRFTGSGSGTDFTFTISSVQAEDLAVYYCQQHYSPPYTFGGGTKLEIKR

SEQ ID NO: 43 - DNA, 324 bp, Mus sp.
  source 1..324: mol_type = unassigned DNA
  CDS 1..324: protein_id = 44; translation = DIVMTQSHKFLSTSVGDRVSITCKASQDVVTAVAWFQQKPGQSP KLLIYSASHRYTGVPDRFTGSGSGTDFTFTIISVQAEDLAVYYCQQHYTTPTTFGGGT KLDFRR
gacattgtgatgacccagtctcacaaattcttgtccacatcagttggagacagggtcagtatcacctgcaaggccagtcaggatgtggttactgctgttgcctggtttcaacagaaaccaggacaatctccaaaactactgatttattcggcatcccaccggtacactggagtccctgatcgcttcactggcagtggatctgggacagatttcactttcaccatcatcagtgtgcaggctgaagacctggcagtttattactgtcaacaacattatactactcccacgacgttcggtggaggcaccaagctggacttcagacgg

SEQ ID NO: 44 - protein, 108 aa, Mus sp.
DIVMTQSHKFLSTSVGDRVSITCKASQDVVTAVAWFQQKPGQSPKLLIYSASHRYTGVPDRFTGSGSGTDFTFTIISVQAEDLAVYYCQQHYTTPTTFGGGTKLDFRR

SEQ ID NO: 45 - DNA, 324 bp, Mus sp.
  source 1..324: mol_type = unassigned DNA
  CDS 1..324: protein_id = 46; translation = DTVMTQSHKFISTSVGDRVSITCKASQVVGSAVAWYQQKPGQSP KLLIYWASTRHTGVPDRFTGSGSGTDFTLTISNVQSEDLADYFCQQYNSYPYTFGGGT KLEIKR
gacactgtgatgacccagtctcacaaattcatatccacatcagttggagacagggtcagcatcacctgcaaggccagtcaggttgtgggtagtgctgtagcctggtatcaacagaaaccagggcaatctcctaaactactgatttactgggcatccacccggcacactggagtccctgatcgcttcacaggcagtggatctgggacagatttcactctcaccattagcaatgtgcagtctgaagacttggcagattatttctgtcagcaatataacagctatccgtacacgttcggaggggggaccaagctggaaataaaacgg

SEQ ID NO: 46 - protein, 108 aa, Mus sp.
DTVMTQSHKFISTSVGDRVSITCKASQVVGSAVAWYQQKPGQSPKLLIYWASTRHTGVPDRFTGSGSGTDFTLTISNVQSEDLADYFCQQYNSYPYTFGGGTKLEIKR

SEQ ID NO: 47 - DNA, 324 bp, Mus sp.
  source 1..324: mol_type = unassigned DNA
  CDS 1..324: protein_id = 48; translation = DIVMTQSQKFMSTSVGDRVSVTCKASQNVGTNVAWYQHKPGQSP KIMIYSASSRYSGVPDRFTGSGSGTLFTLTINNVQSEDLAEYFCQQYNSYPLTFGSGT KLEIKR
gacattgtgatgacccagtctcaaaaattcatgtccacatcagtaggagacagggtcagcgtcacctgcaaggccagtcagaatgtgggtactaatgttgcctggtatcaacacaaaccaggacaatcccctaaaataatgatttattcggcgtcctcccggtacagtggagtccctgatcgcttcacaggcagtggatctgggacacttttcactctcaccatcaacaatgtgcagtctgaagacttggcagagtatttctgtcagcaatataacagctatcctctcacgttcggctcggggacaaagttggaaataaaacgg

SEQ ID NO: 48 - protein, 108 aa, Mus sp.
DIVMTQSQKFMSTSVGDRVSVTCKASQNVGTNVAWYQHKPGQSPKIMIYSASSRYSGVPDRFTGSGSGTLFTLTINNVQSEDLAEYFCQQYNSYPLTFGSGTKLEIKR

SEQ ID NO: 49 - DNA, 342 bp, Mus sp.
  source 1..342: mol_type = unassigned DNA
  CDS 1..342: protein_id = 50; translation = QIQLVQSGPELKKPGETVKISCKASGYTLTSYGMNWVKQAPGKG LKWMGWINTYTGEPTYADDFKGRFAFSLETSASTAFLQINNLKNEDTATYFCVRRGFA YWGQGTLVTVSA
cagatccagttggtgcagtctggacctgagctgaagaagcctggagagacagtcaagatctcctgcaaggcttctgggtataccctcacaagctacggaatgaactgggtgaagcaggctccaggaaagggtttaaagtggatgggctggataaacacctacactggagaaccaacatatgctgatgactttaagggacgttttgccttctctttggaaacctctgccagcactgcctttttgcagatcaacaacctcaaaaatgaggacacggctacatatttctgtgtaagacgcgggtttgcttactggggccaagggactctggtcactgtctctgca

SEQ ID NO: 50 - protein, 114 aa, Mus sp.
QIQLVQSGPELKKPGETVKISCKASGYTLTSYGMNWVKQAPGKGLKWMGWINTYTGEPTYADDFKGRFAFSLETSASTAFLQINNLKNEDTATYFCVRRGFAYWGQGTLVTVSA

SEQ ID NO: 51 - DNA, 342 bp, Mus sp.
  source 1..342: mol_type = unassigned DNA
  CDS 1..342: protein_id = 52; translation = QIQLVQSGPELKKPGETVKISCKASGYTFTNSGMNWVKQAPGKG LKWMGWINTYTGEPTYADDFKGRFAFSLETSASSAYLQISNLKNEDTATYFCARRGFV YWGQGTLVTVSA
cagatccagttggtgcagtctggacctgagctgaagaagcctggagagacagtcaagatctcctgcaaggcttctgggtataccttcacaaactctggaatgaactgggtgaagcaggctccaggaaagggtttaaagtggatgggctggataaacacctacactggagagccgacatatgctgatgacttcaagggacggtttgccttctctttggaaacctctgccagctctgcctatttgcagatcagtaacctcaaaaatgaggacacggctacatatttctgtgcaagaaggggttttgtttactggggccaagggactctggtaactgtctctgca

SEQ ID NO: 52 - protein, 114 aa, Mus sp.
QIQLVQSGPELKKPGETVKISCKASGYTFTNSGMNWVKQAPGKGLKWMGWINTYTGEPTYADDFKGRFAFSLETSASSAYLQISNLKNEDTATYFCARRGFVYWGQGTLVTVSA

SEQ ID NO: 53 - DNA, 360 bp, Mus sp.
  source 1..360: mol_type = unassigned DNA
  CDS 1..360: protein_id = 54; translation = QVQLQQSGAELARPGTSVKLSCKASGYTFTDYWMQWVKQRPGQG LEWIGTIYPGDGDTGYAQKFKGKATLTADKSSKTVYMHLSSLASEDSAVYYCARGDYY GSNSLDYWGQGTSVTVSS
caggttcagctccagcagtctggggctgagctggcaagacctgggacttcagtgaagttgtcctgtaaggcttctggctacacctttactgactactggatgcagtgggtaaaacagaggcctggacagggtctggagtggattgggactatttatcctggagatggtgatactgggtacgctcagaagttcaagggcaaggccacattgactgcggataaatcctccaaaacagtctacatgcacctcagcagtttggcttctgaggactctgcggtctattactgtgcaagaggggattactacggtagtaattctttggactattggggtcaaggaacctcagtcaccgtctcctca

SEQ ID NO: 54 - protein, 120 aa, Mus sp.
QVQLQQSGAELARPGTSVKLSCKASGYTFTDYWMQWVKQRPGQGLEWIGTIYPGDGDTGYAQKFKGKATLTADKSSKTVYMHLSSLASEDSAVYYCARGDYYGSNSLDYWGQGTSVTVSS

SEQ ID NO: 55 - DNA, 357 bp, Mus sp.
  source 1..357: mol_type = unassigned DNA
  CDS 1..357: protein_id = 56; translation = QVQLQQSGPELVRPGASVKISCKTSGYAFSGSWMNWVKQRPGQG LEWIGRIYPGDGDIIYNGNFRDKVTLSADKSSNTAYMQLSSLTSVDSAVYFCSRWGTF TPSFDYWGQGTTLTVSS
caggtccagttacagcaatctggacctgaactggtgaggcctggggcctcagtgaagatttcctgcaaaacttctggctacgcattcagtggctcctggatgaactgggtgaagcagaggcctggacagggtctagagtggattggacggatttatccgggagatggagatatcatttacaatgggaatttcagggacaaggtcacactgtctgcagacaaatcctccaacacagcctacatgcagctcagcagcctgacctctgtggactctgcggtctatttttgttcgagatgggggacatttacgccgagttttgactattggggccaaggcaccactctcacagtctcctca

SEQ ID NO: 56 - protein, 119 aa, Mus sp.
QVQLQQSGPELVRPGASVKISCKTSGYAFSGSWMNWVKQRPGQGLEWIGRIYPGDGDIIYNGNFRDKVTLSADKSSNTAYMQLSSLTSVDSAVYFCSRWGTFTPSFDYWGQGTTLTVSS

SEQ ID NO: 57 - DNA, 351 bp, Mus sp.
  source 1..351: mol_type = unassigned DNA
  CDS 1..351: protein_id = 58; translation = DVKLVESGGGLVKPGGSLKLSCEASGFTFSSYTLSWVRQTPETR LEWVATISIGGRYTYYPDSVEGRFTISRDNAKNTLYLQMNSLKSEDTAMYYCTRDFNG YSDFWGQGTTLTVSS
gacgtgaagctggtggagtctgggggaggcttagtgaagcctggagggtccctgaaactctcctgtgaagcctctggattcactttcagtagctataccctgtcttgggttcgccagactccggagacgaggctggagtgggtcgcaaccattagtattggtggtcgctacacctattatccagacagtgtggagggccgattcaccatctccagagacaatgccaagaacaccctgtacctgcaaatgaacagtctgaagtctgaggacacagccatgtattactgtacaagagattttaatggttactctgacttctggggccaaggcaccactctcacagtctcctca

SEQ ID NO: 58 - protein, 117 aa, Mus sp.
DVKLVESGGGLVKPGGSLKLSCEASGFTFSSYTLSWVRQTPETRLEWVATISIGGRYTYYPDSVEGRFTISRDNAKNTLYLQMNSLKSEDTAMYYCTRDFNGYSDFWGQGTTLTVSS

SEQ ID NO: 59 - DNA, 360 bp, Mus sp.
  source 1..360: mol_type = unassigned DNA
  CDS 1..360: protein_id = 60; translation = NVQLVESGGGLVQPGGSRKLSCAASGFTFSNFGMHWVRQAPEKG LEWVAYIRSGSGTIYYSDTVKGRFTISRDNPKNTLFLQMTSLRSEDTAMYYCARSYYD FGAWFAYWGQGTLVTVSA
aatgtacagctggtagagtctgggggaggcttagtgcagcctggagggtcccggaaactctcctgtgcagcctctggattcactttcagtaactttggaatgcactgggttcgtcaggctccagagaagggtctggagtgggtcgcatacattcgtagtggcagtggtaccatctactattcagacacagtgaagggccgattcaccatctccagagacaatcccaagaacaccctgttcctgcaaatgaccagtctaaggtctgaggacacggccatgtattactgtgcaagatcctactatgatttcggggcctggtttgcttactggggccaagggactctggtcactgtctctgca

SEQ ID NO: 60 - protein, 120 aa, Mus sp.
NVQLVESGGGLVQPGGSRKLSCAASGFTFSNFGMHWVRQAPEKGLEWVAYIRSGSGTIYYSDTVKGRFTISRDNPKNTLFLQMTSLRSEDTAMYYCARSYYDFGAWFAYWGQGTLVTVSA

SEQ ID NO: 61 - DNA, 324 bp, Homo sapiens
  source 1..324: mol_type = unassigned DNA
  CDS 1..324: protein_id = 62; translation = DIVMTQSHLSMSTSLGDPVSITCKASQDVSTVVAWYQQKPGQSP RRLIYSASYRYIGVPDRFTGSGAGTDFTFTISSVQAEDLAVYYCQQHYSPPYTFGGGT KLEIKR
gatatcgtaatgacccagtcccacctgagtatgagtacctccctgggagatcctgtgtcaatcacttgcaaggcctcacaggatgtgagcaccgtcgttgcttggtatcagcagaagcccgggcaatcacccagacgtctcatctactcagcatcataccgttacatcggggtgcctgaccgatttactggctctggcgctggcacagatttcacctttacaattagttccgtccaggccgaagacctggccgtgtactactgccagcagcactacagtcccccatacactttcgggggagggactaagctcgaaatcaaacgt

SEQ ID NO: 62 - protein, 108 aa, Homo sapiens
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 63 - DNA, 324 bp, Homo sapiens
  source 1..324: mol_type = unassigned DNA
  CDS 1..324: protein_id = 64; translation = DIVMAQSHLSMSTSLGDPVSITCKASQDVSTVVAWYQQKPGQSP RRLIYSASYRYIGVPDRFTGSGAGTDFTFTISSVQAEDLAVYYCQQHYSPPYTFGGGT KLEIKR
gacattgttatggctcaaagccatctgtctatgagcacatctctgggagatcctgtgtccatcacttgcaaagccagtcaagacgtgtctacagttgttgcatggtatcaacagaagccaggccagtcacccagacggctcatttactcagcttcttaccgatacatcggggtccctgacagatttacaggtagtggggccggtactgacttcacttttactatctcatccgtacaagccgaagacctggcagtatattactgccagcaacattattccccaccctacacattcggcgggggtactaagctggaaattaaacgt

SEQ ID NO: 64 - protein, 108 aa, Homo sapiens
DIVMAQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKR

SEQ ID NO: 65 - DNA, 360 bp, Homo sapiens
  source 1..360: mol_type = unassigned DNA
  CDS 1..360: protein_id = 66
caggtacagctcgttcagtccggcgccgaggtagctaagcctggtacttccgtaaaattgtcctgtaaggcttccgggtacacatttacagactactggatgcagtgggtaaaacagcggccaggtcagggcctggagtggattggaacaatatatcccggcgacggcgacacaggctatgcccagaagtttcaaggcaaggcaacccttactgctgataaatcttccaagactgtctacatgcatctgtcttccttggcatctgaggatagcgctgtctattactgtgctaggggggactactatgggtcaaattccctggattactggggccagggcaccagtgtcaccgtgagcagc

SEQ ID NO: 66 - protein, 120 aa, Homo sapiens [sequence omitted]

SEQ ID NO: 67 - DNA, 324 bp, Homo sapiens
  source 1..324: mol_type = unassigned DNA
  CDS 1..324: protein_id = 68; translation = DTVMTQSPSTISTSVGDRVSITCKASQVVGSAVAWYQQKPGQSP KLLIYWASTRHTGVPDRFTGSGSGTDFTLTISNVQSDDLADYFCQQYNSYPYTFGGGT KLEIKR
gacaccgtgatgacccagtccccctccaccatctccacctctgtgggcgaccgggtgtccatcacctgtaaggcctcccaggtggtgggctccgccgtggcctggtatcagcagaagcctggccagtcccctaagctgctgatctactgggcctccacccggcataccggcgtgcctgaccggttcaccggctccggcagcggcaccgacttcaccctgaccatctccaacgtgcagtccgacgacctggccgactacttctgccagcagtacaactcctacccttacacctttggcggcggaacaaagctggagatcaagcgt

SEQ ID NO: 68 - protein, 108 aa, Homo sapiens
DTVMTQSPSTISTSVGDRVSITCKASQVVGSAVAWYQQKPGQSPKLLIYWASTRHTGVPDRFTGSGSGTDFTLTISNVQSDDLADYFCQQYNSYPYTFGGGTKLEIKR

SEQ ID NO: 69 - DNA, 324 bp, Homo sapiens
  source 1..324: mol_type = unassigned DNA
  CDS 1..324: protein_id = 70; translation = DTVMTQSPSSISTSIGDRVSITCKASQVVGSAVAWYQQKPGQSP KLLIYWASTRHTGVPARFTGSGSGTDFTLTISNVQSEDLADYFCQQYNSYPYTFGGGT KLEIKR
gacaccgtgatgacccagtccccctcctccatctccacctccatcggcgaccgggtgtccatcacctgtaaggcctcccaggtggtgggctccgccgtggcctggtatcagcagaagcctggccagtcccctaagctgctgatctactgggcctccacccggcataccggcgtgcctgcccggttcaccggctccggcagcggcaccgacttcaccctgaccatctccaacgtgcagtccgaggacctggccgactacttctgccagcagtacaactcctacccttacacctttggcggcggaacaaagctggagatcaagcgt

SEQ ID NO: 70 - protein, 108 aa, Homo sapiens
DTVMTQSPSSISTSIGDRVSITCKASQVVGSAVAWYQQKPGQSPKLLIYWASTRHTGVPARFTGSGSGTDFTLTISNVQSEDLADYFCQQYNSYPYTFGGGTKLEIKR

SEQ ID NO: 71 - DNA, 351 bp, Homo sapiens
  source 1..351: mol_type = unassigned DNA
  CDS 1..351: protein_id = 72; translation = EVQLVESGGGLVKPGGSLRLSCEASGFTFSSYTLSWVRQTPGKG LEWVATISIGGRYTYYPDSVKGRFTISRDNAKNTLYLQMNSLKSEDTAMYYCTRDFNG YSDFWGQGTTLTVSS
gaggtgcagctggtggagtctggcggcggactggtgaagcctggcggctccctgaggctgtcctgtgaggcctccggcttcaccttctcctcctacaccctgtcctgggtgaggcagacccctggcaagggcctggagtgggtggccaccatctccatcggcggcaggtacacctactaccctgactccgtgaagggccggttcaccatctcccgggacaacgccaagaacaccctgtacctgcagatgaactccctgaagtccgaggacaccgccatgtactactgtacccgggacttcaacggctactccgacttctggggccagggcaccacactgaccgtgtcctcc

SEQ ID NO: 72 - protein, 117 aa, Homo sapiens
EVQLVESGGGLVKPGGSLRLSCEASGFTFSSYTLSWVRQTPGKGLEWVATISIGGRYTYYPDSVKGRFTISRDNAKNTLYLQMNSLKSEDTAMYYCTRDFNGYSDFWGQGTTLTVSS

SEQ ID NO: 73 - DNA, 36 bp, Mus sp.
  source 1..36: mol_type = unassigned DNA
ggaggatccatagacagatgggggtgtcgttttggc

SEQ ID NO: 74 - DNA, 32 bp, Mus sp.
  source 1..32: mol_type = unassigned DNA
ggaggatcccttgaccaggcatcctagagtca

SEQ ID NO: 75 - DNA, 32 bp, Mus sp.
  source 1..32: mol_type = unassigned DNA
cttccggaattcsargtnmagctgsagsagtc

SEQ ID NO: 76 - DNA, 35 bp, Mus sp.
  source 1..35: mol_type = unassigned DNA
cttccggaattcsargtnmagctgsagsagtcwgg

SEQ ID NO: 77 - DNA, 31 bp, Mus sp.
  source 1..31: mol_type = unassigned DNA
ggagctcgayattgtgmtsacmcarwctmca

SEQ ID NO: 78 - DNA, 46 bp, Mus sp.
  source 1..46: mol_type = unassigned DNA
tatagagctcaagcttggatggtgggaagatggatacagttggtgc

SEQ ID NO: 79 - DNA, 21 bp, Mus sp.
  source 1..21: mol_type = unassigned DNA
atggagtcacagattcaggtc

SEQ ID NO: 80 - DNA, 32 bp, Mus sp.
  source 1..32: mol_type = unassigned DNA
ttttgaattccagtaacttcaggtgtccactc

SEQ ID NO: 81 - protein, 17 aa, Homo sapiens
TIYPGDGDTGYAQKFQG